(19) United States
(12) Patent Application Publication
(10) Pub. No.: US 2025/0018193 A1
(43) Pub. Date: Jan. 16, 2025

(54) CEREBRAL BLOODFLOW ENHANCEMENT

(71) Applicants: Yossi GROSS, Moshav Mazor (IL); Ofri Vaisman, Sunnyvale, CA (US)

(72) Inventors: Yossi GROSS, Moshav Mazor (IL); Ofri Vaisman, Sunnyvale, CA (US)

(21) Appl. No.: 18/229,379

(22) Filed: Aug. 2, 2023

Related U.S. Application Data
(63) Continuation-in-part of application No. 18/351,247, filed on Jul. 12, 2023.

(51) Int. Cl. A61N 1/36

(52) U.S. Cl. CPC A61N 1/36082; A61N 1/36171

(57) ABSTRACT

A method is provided that includes inserting a neurostimulator into a face of a subject and advancing the neurostimulator between a mandibular notch and a zygomatic process of a temporal bone toward a sphenopalatine ganglion (SPG), the subject identified as at risk of or suffering from dementia. Cerebral blood flow (CBF) of the subject is increased by activating the neurostimulator to drive, between first and second SPG-stimulation electrodes of the neurostimulator, alternating current configured to stimulate the SPG. Other embodiments are also described.

### CROSS-REFERENCE TO RELATED APPLICATIONS

The present application is a continuation-in-part of U.S. application Ser. No. 18/351,247, filed Jul. 12, 2023, which is incorporated herein by reference.

### FIELD OF THE APPLICATION

The present invention relates generally to treatment and prevention of Alzheimer's disease, and specifically to electrical and mechanical techniques for treating, preventing, or slowing the progression of Alzheimer's disease.

### BACKGROUND OF THE APPLICATION

Alzheimer's disease is a chronic neurodegenerative disease that causes dementia. Accumulation of substances such as amyloid beta and/or tau protein in the brain is widely believed to contribute to the development of Alzheimer's disease.

The onset of Alzheimer's disease is believed to begin a decade or even two decades before symptoms appear. In the pre-clinical stages, before cognitive symptoms can be detected, multiple pathologies are already present, such as decreased cerebral blood flow, accumulation of amyloid beta (Aβ) plaques in the extracellular spaces, phosphorylated tau tangles within the neurons, chronic inflammation, and reduced gamma oscillations power and synchronization. These interrelated pathologies create a vicious disease cycle resulting in progressive brain degeneration, atrophy and shrinkage.

The first areas of the brain that are prone to damage are the hippocampus and the entorhinal cortex, brain areas involved in learning and memory. As the disease progresses, additional parts of the brain are affected and begin to shrink. By the final stage of Alzheimer's, damage is widespread and brain tissue has shrunk significantly.

U.S. Pat. No. 9,616,221 to Gross describes a method that includes disposing midplane treatment electrodes over a superior sagittal sinus, outside and in electrical contact with a skull of a head of a subject identified as at risk of or suffering from Alzheimer's disease. Lateral treatment electrodes are disposed between 1 and 12 cm of a sagittal midplane of the skull. The subject is treated by electroosmotically driving fluid from a subarachnoid space to the superior sagittal sinus, by activating control circuitry to apply one or more treatment currents between (a) one or more of the midplane treatment electrodes and (b) one or sore of the lateral treatment electrodes. Other embodiments are also described.

U.S. Pat. No. 10,569,086 to Fostick et al. describes a method that includes positioning electrodes in or in contact with the head of a subject identified as in need of enhanced microglial cell activation. Control circuitry is activated to drive the electrodes to apply an electrical current to the brain of the subject, and to configure the electrical current to enhance microglial cell activation for taking up a substance from brain parenchyma. Other embodiments are also described.

U.S. Pat. No. 7,640,062 to Shalev describes a method for treating Alzheimer's disease (AD). The method includes stimulating a sphenopalatine ganglion (SPG) of a subject so that the concentration of a substance in a brain of the subject changes.

U.S. Pat. No. 9,233,245 to Lamensdorf et al. describes a method for treating a subject, including applying electrical stimulation to a site of the subject selected from the group consisting of: a sphenopalatine ganglion (SPG), a greater palatine nerve, a lesser palatine nerve, a sphenopalatine nerve, a communicating branch between a maxillary nerve and an SPG, an otic ganglion, an afferent fiber going into the otic ganglion, an efferent fiber going out of the otic ganglion, an infraorbital nerve, a vidian nerve, a greater superficial petrosal nerve, and a lesser deep petrosal nerve. The stimulation is configured to excite nervous tissue of the site at a strength sufficient to induce at least one neuroprotective occurrence selected from the group consisting of: an increase in cerebral blood flow (CBF) of the subject, and a release of one or more neuroprotective substances, and insufficient to induce a significant increase in permeability of a blood-brain barrier (BBB) of the subject.

McDermott B et al., in “Gamma Band Neural Stimulation in Humans and the Promise of a New Modality to Prevent and Treat Alzheimer's Disease,” J Alzheimers Dis. 2018; 65(2):363-392, report that “Existing treatments for Alzheimer's disease (AD) have questionable efficacy with a need for research into new and more effective therapies to both treat and possibly prevent the condition. This review examines a novel therapeutic modality that shows promise for treating AD based on modulating neuronal activity in the gamma frequency band through external brain stimulation. The gamma frequency band is roughly defined as being between 30 Hz-100 Hz, with the 40 Hz point being of particular significance. The epidemiology, diagnostics, existing pathological models, and related current treatment targets are initially briefly reviewed. Next, the concept of external simulation triggering brain activity in the gamma band with potential demonstration of benefit in AD is introduced with reference to a recent important study using a mouse model of the disease. The review then presents a selection of relevant studies that describe the neurophysiology involved in brain stimulation by external sources, followed by studies involving application of the modality to clinical scenarios. A table summarizing the results of clinical studies applied to AD patients is also reported and may aid future development of the modality. The use of a therapy based on modulation of gamma neuronal activity represents a novel non-invasive, non-pharmacological approach to AD. Although use in clinical scenarios is still a relatively recent area of research, the technique shows good signs of efficacy and may represent an important option for treating AD in the future” (Abstract).

### SUMMARY OF THE APPLICATION

In some embodiments of the present invention, a system is provided for applying multimodal stimulation for treatment and prevention of dementia, such as Alzheimer's disease, by addressing multiple dementia pathologies and inhibiting the brain degeneration cascade that results in cognitive and functional decline. The stimulation treatment modalities are typically non-pharmacological and are non-invasive in some configurations. The stimulation is typically applied to a subject identified as at risk of or suffering from dementia, such as Alzheimer's disease.

In some embodiments of the present invention, the system implements two or more of the stimulation treatment modalities summarized in Table 1:

TABLE 1

| | | Therapeutic |
|---|---|---|
| | | Mechanism(s) and |
| Stimulation | | Expected Clinical |
| Modality | Description | Benefit |
| Sphenopalatine | Alternating current electrical | Promotes secretion of |
| ganglion (SPG) | stimulation of the SPG | nitric oxide (NO), |
| stimulation | | promoting vasodilation, |
| | | increased cerebral blood |
| | | flow, and modulates |
| | | amyloid beta metabolism |
| Transcranial | Non-invasive electrical | Modules microglial cell |
| direct | stimulation that modulates cell | activity, promotes |
| current | activity by changing | metabolic-waste |
| stimulation | membrane voltage using a | clearance and amyloid |
| (tDCS) | weak direct current | beta load reduction |
| Gamma band | Auditory, somatosensory | Promotes oscillatory |
| entrainment | (e.g., vibrational), | neuron synchronization |
| | or transcranial | and connectivity and |
| | Alternating Current | increases EEG gamma |
| | Stimulation (tACS) that | power |
| | modulates oscillatory | |
| | brain activity | |

Alternatively, in some embodiments of the present invention, a system is provided that applies only a single one of these treatment modalities.

There is therefore provided, in accordance with an Inventive Concept 1 of the present invention, a method including:
stimulation electrode to a first SPG-stimulation site of a subject on an external surface of skin between a mandibular notch and a zygomatic process of a temporal bone, the subject identified as at risk of or suffering from dementia;
applying a second SPG-stimulation electrode to a second SPG-stimulation site of the subject; and
increasing cerebral blood flow (CBF) of the subject by activating circuitry to drive, between the first non-invasive SPG-stimulation electrode and the second SPG-stimulation electrode, alternating current configured to stimulate an SPG of the subject.
   Inventive Concept 2. The method according to Inventive Concept 1,
wherein the dementia is Alzheimer's disease, and
wherein non-invasively applying the first non-invasive SPG-stimulation electrode includes non-invasively applying the first non-invasive SPG-stimulation electrode to the first SPG-stimulation site of the subject identified as at risk of or suffering from Alzheimer's disease.
   Inventive Concept 3. The method according to Inventive Concept 1, wherein applying the second SPG-stimulation electrode includes non-invasively applying the second SPG-stimulation electrode to the second SPG-stimulation site.
   Inventive Concept 4. The method according to Inventive Concept 3,
wherein the mandibular notch and the zygomatic process of the temporal bone are a left mandibular notch and a left zygomatic process of a left temporal bone, respectively,
wherein the first SPG-stimulation site is on the external surface of the skin between the left mandibular notch and the left zygomatic process of the left temporal bone, and
wherein the second SPG-stimulation site is on the external surface of the skin between a right mandibular notch and a right zygomatic process of a right temporal bone.
   Inventive Concept 5. The method according to Inventive Concept 3,
wherein the second SPG-stimulation site is within an oral cavity in a vicinity of a greater palatine foramen, and
wherein non-invasively applying the second SPG-stimulation electrode to the second SPG-stimulation site includes placing the second SPG-stimulation electrode in the oral cavity in the vicinity of the greater palatine foramen.
   Inventive Concept 6. The method according to Inventive Concept 1, wherein activating the circuitry includes activating the circuitry to drive the alternating current in pulses having a pulse width of 50-5,000 microseconds and a frequency of 10-60 Hz, e.g., 40 Hz.
   Inventive Concept 7. The method according to Inventive Concept 1, wherein activating the circuitry includes activating the circuitry to drive the alternating current at a frequency of 10-60 Hz, e.g., 10-40 Hz.
   Inventive Concept 8. The method according to Inventive Concept 1, wherein activating the circuitry includes activating the circuitry to drive the alternating current in pulses having a pulse width of 50-5,000 microseconds.
   Inventive Concept 9. The method according to Inventive Concept 1,
wherein the second SPG-stimulation site is within an oral cavity in a vicinity of a greater palatine foramen, and
wherein applying the second SPG-stimulation electrode to the second SPG-stimulation site includes non-invasively placing the second SPG-stimulation electrode in the oral cavity in the vicinity of the greater palatine foramen.
   Inventive Concept 10. The method according to Inventive Concept 9,
wherein an oral support is coupled to the second SPG-stimulation electrode, and
wherein non-invasively applying the second SPG-stimulation electrode to the second SPG-stimulation site includes placing the oral support within the oral cavity such that the oral support non-invasively applies the second SPG-stimulation electrode to the second SPG-stimulation site.
   Inventive Concept 11. The method according to Inventive Concept 10,
wherein the oral support is a dental guard configured to cover maxillary teeth, and
wherein placing the oral support within the oral cavity includes placing the dental guard on the maxillary teeth.
   Inventive Concept 12. The method according to Inventive Concept 10,
wherein the oral support is a gingival support band, and
wherein placing the oral support within the oral cavity includes placing the gingival support band between mucosa of bone supporting maxillary teeth and labial mucosa.
   Inventive Concept 13. The method according to Inventive Concept 10,
wherein the oral support is a maxillary orthodontic retainer, and
wherein non-invasively applying the second SPG-stimulation electrode to the second SPG-stimulation site includes coupling the maxillary orthodontic retainer to maxillary teeth.
   Inventive Concept 14. The method according to Inventive Concept 9,
wherein the method further includes minimally invasively inserting a wire, including both ends thereof, into a greater palatine canal via the greater palatine foramen, and
wherein placing the second SPG-stimulation electrode in the oral cavity in the vicinity of the greater palatine foramen includes placing the second SPG-stimulation electrode in the oral cavity in the vicinity of the greater palatine foramen, such that the second SPG-stimulation electrode is separated from the wire by masticatory mucosa.
   Inventive Concept 15. The method according to Inventive Concept 9, wherein the first SPG-stimulation site and the greater palatine foramen are ipsilateral.
   Inventive Concept 16. The method according to Inventive Concept 1, wherein non-invasively applying the first non-invasive SPG-stimulation electrode to the first SPG-stimulation site includes coupling a head support to a head of the subject such that the head support non-invasively applies the first non-invasive SPG-stimulation electrode to the first SPG-stimulation site.
   Inventive Concept 17. The method according to Inventive Concept 16, wherein applying the second SPG-stimulation electrode includes non-invasively applying the second SPG-stimulation electrode to the second SPG-stimulation site.
   Inventive Concept 18. The method according to Inventive Concept 16,
wherein the head support includes a headphone, which includes one or two earphones, which include one or more speakers,
wherein coupling the head support to the head includes coupling the headphone to the head such that the headphone positions the one or two earphones over or in one or two respective ears of the subject, and
wherein activating the circuitry includes activating the circuitry to drive the one or more speakers of the one or two earphones to apply, to the one or two ears, auditory stimulation that modulates oscillatory brain activity of the subject.
   Inventive Concept 19. The method according to Inventive Concept 18, wherein activating the circuitry includes activating the circuitry to drive the one or more speakers of the one or two earphones to apply the auditory stimulation at a frequency of 30-60 Hz.
   Inventive Concept 20. The method according to Inventive Concept 16,
wherein the head support is coupled to first and second non-invasive transcranial Direct Current Stimulation (tDCS) electrodes,
wherein coupling the head support to the head includes coupling the head support to the head such that the head support applies:
   the first non-invasive tDCS electrode to the external surface of the skin at a first tDCS site on a temple of the subject, and
   the second non-invasive tDCS electrode to the external surface of the skin at a non-cephalic second tDCS site, and
   wherein activating the circuitry includes activating the circuitry to drive, between the first and the second non-invasive tDCS electrodes, direct current configured to promote amyloid beta clearance from a brain of the subject.
      Inventive Concept 21. The method according to Inventive Concept 20, wherein coupling the head support to the head includes coupling the head support to the head such that the head support applies the non-cephalic second non-invasive tDCS electrode to the external surface of the skin on a part of the subject selected from the group consisting of: a neck and a shoulder.
      Inventive Concept 22. The method according to Inventive Concept 21, wherein coupling the head support to the head includes coupling the head support to the head such that the head support applies the non-cephalic second non-invasive tDCS electrode to the external surface of a nape of the subject.
      Inventive Concept 23. The method according to Inventive Concept 20, wherein activating the circuitry includes activating the circuitry to drive the direct current at an amplitude of 1-5 milliamps.
      Inventive Concept 24. The method according to Inventive Concept 1,
wherein the second SPG-stimulation site is outside a nasal cavity of the subject, and
wherein applying the second SPG-stimulation electrode includes non-invasively applying the second SPG-stimulation electrode to the second SPG-stimulation site outside the nasal cavity.

There is further provided, in accordance with an Inventive Concept 25 of the present invention, a method including:
non-invasively applying a first non-invasive sphenopalatine ganglion (SPG)-stimulation electrode to a first SPG-stimulation site of a subject on an external surface of skin lateral to a nose, medial to a zygomatic bone, and inferior to an infraorbital margin, the subject identified as at risk of or suffering from dementia;
applying a second SPG-stimulation electrode to a second SPG-stimulation site of the subject; and
increasing cerebral blood flow (CBF) of the subject by activating circuitry to drive, between the first non-invasive SPG-stimulation electrode and the second SPG-stimulation electrode, alternating current configured to stimulate an SPG of the subject.
   Inventive Concept 26. The method according to Inventive Concept 25,
wherein the dementia is Alzheimer's disease, and
wherein non-invasively applying the first non-invasive SPG-stimulation electrode includes non-invasively applying the first non-invasive SPG-stimulation electrode to the first SPG-stimulation site of the subject identified as at risk of or suffering from Alzheimer's disease.
   Inventive Concept 27. The method according to Inventive Concept 25, wherein the first SPG-stimulation site is medial to a plane that is parallel to a sagittal plane of a body of the subject and includes an infraorbital foramen.
   Inventive Concept 28. The method according to Inventive Concept 25, wherein applying the second SPG-stimulation electrode includes non-invasively applying the second SPG-stimulation electrode to the second SPG-stimulation site.
   Inventive Concept 29. The method according to Inventive Concept 28, wherein the second SPG-stimulation site is on the external surface of the skin (a) over an angle, a ramus, or a neck of a mandible, or (b) on a neck of the subject.
   Inventive Concept 30. The method according to Inventive Concept 29, wherein the second SPG-stimulation site is on the external surface of the skin over the angle, the ramus, or the neck of the mandible.
   Inventive Concept 31. The method according to Inventive Concept 30, wherein the second SPG-stimulation site is on the external surface of the skin over a posterior third of the angle, the ramus, or the neck of the mandible.
   Inventive Concept 32. The method according to Inventive Concept 29, wherein the second SPG-stimulation site is on the external surface of the skin on the neck of the subject.
   Inventive Concept 33. The method according to Inventive Concept 32, wherein the
second SPG-stimulation site is on the external surface of the skin on the neck of the subject near the mandible.
   Inventive Concept 34. The method according to Inventive Concept 28,
wherein the second SPG-stimulation site is within an oral cavity in a vicinity of a greater palatine foramen, and
wherein non-invasively applying the second SPG-stimulation electrode to the second SPG-stimulation site includes placing the second SPG-stimulation electrode in the oral cavity in the vicinity of the greater palatine foramen.
   Inventive Concept 35. The method according to Inventive Concept 25, wherein activating the circuitry includes activating the circuitry to drive the alternating current in pulses having a pulse width of 50-5,000 microseconds and a frequency of 10-60 Hz, e.g., 10-40 Hz.
   Inventive Concept 36. The method according to Inventive Concept 25, wherein activating the circuitry includes activating the circuitry to drive the alternating current at a frequency of 10-60 Hz, e.g., 10-40 Hz.
   Inventive Concept 37. The method according to Inventive Concept 25, wherein activating the circuitry includes activating the circuitry to drive the alternating current in pulses having a pulse width of 50-5,000 microseconds.
   Inventive Concept 38. The method according to Inventive Concept 25,
wherein the second SPG-stimulation site is within an oral cavity in a vicinity of a greater palatine foramen, and
wherein applying the second SPG-stimulation electrode to the second SPG-stimulation site includes non-invasively placing the second SPG-stimulation electrode in the oral cavity in the vicinity of the greater palatine foramen.
   Inventive Concept 39. The method according to Inventive Concept 38,
wherein an oral support is coupled to the second SPG-stimulation electrode, and
wherein non-invasively applying the second SPG-stimulation electrode to the second SPG-stimulation site includes placing the oral support within the oral cavity such that the oral support non-invasively applies the second SPG-stimulation electrode to the second SPG-stimulation site.
   Inventive Concept 40. The method according to Inventive Concept 39,
wherein the oral support is a dental guard configured to cover maxillary teeth, and
wherein placing the oral support within the oral cavity includes placing the dental guard on the maxillary teeth.
   Inventive Concept 41. The method according to Inventive Concept 39,
wherein the oral support is a gingival support band, and
wherein placing the oral support within the oral cavity includes placing the gingival support band between mucosa of bone supporting maxillary teeth and labial mucosa.
   Inventive Concept 42. The method according to Inventive Concept 39,
wherein the oral support is a maxillary orthodontic retainer, and
wherein non-invasively applying the second SPG-stimulation electrode to the second SPG-stimulation site includes coupling the maxillary orthodontic retainer to maxillary teeth.
   Inventive Concept 43. The method according to Inventive Concept 38,
wherein the method further includes minimally invasively inserting a wire, including both ends thereof, into a greater palatine canal via the greater palatine foramen, and
wherein placing the second SPG-stimulation electrode in the oral cavity in the vicinity of the greater palatine foramen includes placing the second SPG-stimulation electrode in the oral cavity in the vicinity of the greater palatine foramen, such that the second SPG-stimulation electrode is separated from the wire by masticatory mucosa.
   Inventive Concept 44. The method according to Inventive Concept 38, wherein the first SPG-stimulation site and the greater palatine foramen are ipsilateral.
   Inventive Concept 45. The method according to Inventive Concept 25, wherein non-invasively applying the first non-invasive SPG-stimulation electrode to the first SPG-stimulation site includes coupling a head support to a head of the subject such that the head support non-invasively applies the first non-invasive SPG-stimulation electrode to the first SPG-stimulation site.
   Inventive Concept 46. The method according to Inventive Concept 45, wherein applying the second SPG-stimulation electrode includes non-invasively applying the second SPG-stimulation electrode to the second SPG-stimulation site.
   Inventive Concept 47. The method according to Inventive Concept 45,
wherein the head support includes a headphone, which includes one or two earphones, which include one or more speakers,
wherein coupling the head support to the head includes coupling the headphone to the head such that the headphone positions the one or two earphones over or in one or two respective ears of the subject, and
wherein activating the circuitry includes activating the circuitry to drive the one or more speakers of the one or two earphones to apply, to the one or two ears, auditory stimulation that modulates oscillatory brain activity of the subject.
   Inventive Concept 48. The method according to Inventive Concept 47, wherein activating the circuitry includes activating the circuitry to drive the one or more speakers of the one or two earphones to apply the auditory stimulation at a frequency of 30-60 Hz.
   Inventive Concept 49. The method according to Inventive Concept 45,
wherein the head support is coupled to first and second non-invasive transcranial Direct Current Stimulation (tDCS) electrodes,
wherein coupling the head support to the head includes coupling the head support to the head such that the head support applies:
   the first non-invasive tDCS electrode to the external surface of the skin at a first tDCS site on a temple of the subject, and
   the second non-invasive tDCS electrode to the external surface of the skin at a non-cephalic second tDCS site, and
   wherein activating the circuitry includes activating the circuitry to drive, between the first and the second non-invasive tDCS electrodes, direct current configured to promote amyloid beta clearance from a brain of the subject.
      Inventive Concept 50. The method according to Inventive Concept 49, wherein coupling the head support to the head includes coupling the head support to the head such that the head support applies the non-cephalic second non-invasive tDCS electrode to the external surface of the skin on a part of the subject selected from the group consisting of: a neck and a shoulder.
      Inventive Concept 51. The method according to Inventive Concept 50, wherein coupling the head support to the head includes coupling the head support to the head such that the head support applies the non-cephalic second non-invasive tDCS electrode to the external surface of a nape of the subject.
      Inventive Concept 52. The method according to Inventive Concept 49, wherein activating the circuitry includes activating the circuitry to drive the direct current at an amplitude of 1-5 milliamps.
      Inventive Concept 53. The method according to Inventive Concept 25,
wherein the second SPG-stimulation site is outside a nasal cavity of the subject, and
wherein applying the second SPG-stimulation electrode includes non-invasively applying the second SPG-stimulation electrode to the second SPG-stimulation site outside the nasal cavity.

There is still further provided, in accordance with an Inventive Concept 54 of the present invention, a method including:
non-invasively applying:
   a first non-invasive sphenopalatine ganglion (SPG)-stimulation electrode to an external surface of skin of a head of a subject at a first SPG-stimulation site, the subject identified as at risk of or suffering from dementia, and
   a second non-invasive SPG-stimulation electrode at a second SPG-stimulation site of the subject, such that a line between the first and the second SPG-stimulation sites passes within 1 cm of an SPG of the subject; and
increasing cerebral blood flow (CBF) of the subject by activating circuitry to drive, between the first and the second non-invasive SPG-stimulation electrodes, alternating current configured to stimulate the SPG.
   Inventive Concept 55. The method according to Inventive Concept 54,
wherein the dementia is Alzheimer's disease, and
wherein non-invasively applying the first non-invasive SPG-stimulation electrode includes non-invasively applying the first non-invasive SPG-stimulation electrode to the external surface of the skin of the head of the subject identified as at risk of or suffering from Alzheimer's disease.
   Inventive Concept 56. The method according to Inventive Concept 54, wherein non-invasively applying the first non-invasive SPG-stimulation electrode includes non-invasively applying the first non-invasive SPG-stimulation electrode to the external surface of the skin of a face of the head at the first SPG-stimulation site.
   Inventive Concept 57. The method according to Inventive Concept 54, wherein non-invasively applying the second non-invasive SPG-stimulation electrode includes non-invasively applying the second non-invasive SPG-stimulation electrode to the external surface of the skin of a face of the head at the second SPG-stimulation site.
   Inventive Concept 58. The method according to Inventive Concept 54,
wherein the second SPG-stimulation site is within an oral cavity in a vicinity of a greater palatine foramen, and
wherein non-invasively applying the second SPG-stimulation electrode to the second SPG-stimulation site includes placing the second SPG-stimulation electrode in the oral cavity in the vicinity of the greater palatine foramen.

There is additionally provided, in accordance with an Inventive Concept 59 of the present invention, apparatus including:
a first non-invasive SPG-stimulation electrode;
a head support, which (a) is coupled to a first non-invasive sphenopalatine ganglion (SPG)-stimulation electrode, and (b) configured to non-invasively apply the first non-invasive SPG-stimulation electrode to a first SPG-stimulation site of a subject on an external surface of skin between a mandibular notch and a zygomatic process of a temporal bone, when the head support is worn on a head of the subject;
a second SPG-stimulation electrode, which is configured to be applied to a second SPG-stimulation site of the subject; and
circuitry, which is configured to increase cerebral blood flow (CBF) of the subject by driving, between the first non-invasive SPG-stimulation electrode and the second SPG-stimulation electrode, alternating current configured to stimulate an SPG of the subject.
   Inventive Concept 60. The apparatus according to Inventive Concept 59, wherein the second SPG-stimulation electrode is non-invasive and is configured to be non-invasively applied to the second SPG-stimulation site.
   Inventive Concept 61. The apparatus according to Inventive Concept 60,
wherein the mandibular notch and the zygomatic process of the temporal bone are a left mandibular notch and a left zygomatic process of a left temporal bone, respectively, and
wherein the head support is (a) coupled to the first and the second non-invasive SPG-stimulation electrodes, and (b) configured to non-invasively apply, when the head support is worn on a head of the subject:
   the first non-invasive SPG-stimulation electrode to the first SPG-stimulation site on the external surface of the skin between the left mandibular notch and the left zygomatic process of the left temporal bone, and
   the second non-invasive SPG-stimulation electrode to the second SPG-stimulation site on the external surface of the skin between a right mandibular notch and a right zygomatic process of a right temporal bone.
      Inventive Concept 62. The apparatus according to Inventive Concept 60,
wherein the second SPG-stimulation site is within an oral cavity in a vicinity of a greater palatine foramen, and
wherein the second non-invasive SPG-stimulation electrode is configured to be placed in the oral cavity in the vicinity of the greater palatine foramen.

There is yet additionally provided, in accordance with an Inventive Concept 63 of the present invention, apparatus including:
a first non-invasive SPG-stimulation electrode;
a head support, which (a) is coupled to a first non-invasive sphenopalatine ganglion (SPG)-stimulation electrode, and (b) configured to non-invasively apply the first non-invasive SPG-stimulation electrode to a first SPG-stimulation site of a subject on an external surface of skin lateral to a nose, medial to a zygomatic bone, and inferior to an infraorbital margin, when the head support is worn on a head of the subject;
a second SPG-stimulation electrode, which is configured to be applied to a second SPG-stimulation site of the subject; and
circuitry, which is configured to increase cerebral blood flow (CBF) of the subject by driving, between the first non-invasive SPG-stimulation electrode and the second SPG-stimulation electrode, alternating current configured to stimulate an SPG of the subject.
   Inventive Concept 64. The apparatus according to Inventive Concept 63,
wherein the first SPG-stimulation site is medial to a plane that is parallel to a sagittal plane of a body of the subject and includes an infraorbital foramen, and
wherein the head support is configured to non-invasively apply the first non-invasive SPG-stimulation electrode to the first SPG-stimulation site lateral to the nose, medial to the plane, and inferior to the infraorbital margin.
   Inventive Concept 65. The apparatus according to Inventive Concept 63, wherein the second SPG-stimulation electrode is non-invasive and is configured to be non-invasively applied to the second SPG-stimulation site.
   Inventive Concept 66. The apparatus according to Inventive Concept 65,
wherein the second SPG-stimulation site is on the external surface of the skin (a) over an angle, a ramus, or a neck of a mandible, or (b) on a neck of the subject, and
wherein the head support is configured to non-invasively apply the first non-invasive SPG-stimulation electrode to the first SPG-stimulation site on the external surface of the skin (a) over the angle, the ramus, or the neck of the mandible, or (b) on the neck of the subject.
   Inventive Concept 67. The apparatus according to Inventive Concept 66,
wherein the second SPG-stimulation site is on the external surface of the skin over the angle, the ramus, or the neck of the mandible, and
wherein the head support is configured to non-invasively apply the first non-invasive SPG-stimulation electrode to the first SPG-stimulation site on the external surface of the skin over the angle, the ramus, or the neck of the mandible.
   Inventive Concept 68. The apparatus according to Inventive Concept 67,
wherein the second SPG-stimulation site is on the external surface of the skin over a posterior third of the angle, the ramus, or the neck of the mandible, and
wherein the head support is configured to non-invasively apply the first non-invasive SPG-stimulation electrode to the first SPG-stimulation site on the external surface of the skin over the posterior third of the angle, the ramus, or the neck of the mandible.
   Inventive Concept 69. The apparatus according to Inventive Concept 66,
wherein the second SPG-stimulation site is on the external surface of the skin on the neck of the subject, and
wherein the head support is configured to non-invasively apply the first non-invasive SPG-stimulation electrode to the first SPG-stimulation site on the external surface of the skin on the neck of the subject.
   Inventive Concept 70. The apparatus according to Inventive Concept 65,
wherein the second SPG-stimulation site is within an oral cavity in a vicinity of a greater palatine foramen, and
wherein the second non-invasive SPG-stimulation electrode is configured to be placed in the oral cavity in the vicinity of the greater palatine foramen.
   Inventive Concept 71. The apparatus according to any one of Inventive Concepts 59 and 63, wherein the circuitry is configured to drive the alternating current in pulses having a pulse width of 50-5,000 microseconds and a frequency of 10-60 Hz, e.g., 10-40 Hz.
   Inventive Concept 72. The apparatus according to any one of Inventive Concepts 59 and 63, wherein the circuitry is configured to drive the alternating current at a frequency of 10-60 Hz, e.g., 10-40 Hz.
   Inventive Concept 73. The apparatus according to any one of Inventive Concepts 59 and 63, wherein the circuitry is configured to drive the alternating current in pulses having a pulse width of 50-5,000 microseconds.
   Inventive Concept 74. The apparatus according to any one of Inventive Concepts 59, 63, and 71-73,
wherein the second SPG-stimulation site is within an oral cavity in a vicinity of a greater palatine foramen, and
wherein the second non-invasive SPG-stimulation electrode is configured to be placed in the oral cavity in the vicinity of the greater palatine foramen.
   Inventive Concept 75. The apparatus according to Inventive Concept 74,
further including an oral support,
wherein the oral support is coupled to the second non-invasive SPG-stimulation electrode, and
wherein the oral support is configured to non-invasively apply the second non-invasive SPG-stimulation electrode to the second SPG-stimulation site when the oral support is placed within the oral cavity.
   Inventive Concept 76. The apparatus according to Inventive Concept 75,
wherein the oral support includes a dental guard configured to cover maxillary teeth, and
wherein the dental guard is configured to non-invasively apply the second non-invasive SPG-stimulation electrode to the second SPG-stimulation site when the dental guard is placed on the maxillary teeth.
   Inventive Concept 77. The apparatus according to Inventive Concept 75,
wherein the oral support includes a gingival support band, and
wherein the gingival support band is configured to non-invasively apply the second non-invasive SPG-stimulation electrode to the second SPG-stimulation site when the gingival support band is placed between mucosa of bone supporting maxillary teeth and labial mucosa.
   Inventive Concept 78. The apparatus according to Inventive Concept 75,
wherein the oral support includes a maxillary orthodontic retainer, and
wherein the maxillary orthodontic retainer is configured to non-invasively apply the second non-invasive SPG-stimulation electrode to the second SPG-stimulation site when the maxillary orthodontic retainer is coupled to maxillary teeth.
   Inventive Concept 79. The apparatus according to Inventive Concept 74, further including a wire, wherein the wire, including both ends thereof, is configured to be minimally invasively inserted into a greater palatine canal via the greater palatine foramen.
   Inventive Concept 80. The apparatus according to any one of Inventive Concepts 59, 63, and 71-73,
wherein the head support includes a headphone, which (a) includes one or two earphones, which include one or more speakers, and (b) is configured to position the one or two earphones over or in one or two respective ears of the subject, and
wherein the circuitry is configured to drive the one or more speakers of the one or two earphones to apply, to the one or two ears, auditory stimulation that modulates oscillatory brain activity.
   Inventive Concept 81. The apparatus according to Inventive Concept 80, wherein the circuitry is configured to drive the one or more speakers of the one or two earphones to apply the auditory stimulation at a frequency of 30-60 Hz.
   Inventive Concept 82. The apparatus according to any one of Inventive Concepts 59, 63, and 71-73,
further including first and second non-invasive transcranial Direct Current Stimulation (tDCS) electrodes, coupled to the head support,
wherein the head support is configured to apply:
   the first non-invasive tDCS electrode to the external surface of the skin at a first tDCS site on a temple of the subject, and
   the second non-invasive tDCS electrode to the external surface of the skin at a non-cephalic second tDCS site, and
wherein the circuitry is configured to drive, between the first and the second tDCS electrodes, direct current configured to promote amyloid beta clearance from a brain of the subject.
   Inventive Concept 83. The apparatus according to Inventive Concept 82, wherein the head support is configured to apply the second non-invasive tDCS electrode to the external surface of the skin on a part of the subject selected from the group consisting of: a neck and a shoulder.
   Inventive Concept 84. The apparatus according to Inventive Concept 83, wherein the head support is configured to apply the second non-invasive tDCS electrode to the external surface of a nape of the subject.
   Inventive Concept 85. The apparatus according to Inventive Concept 82, wherein the circuitry is configured to drive the direct current at an amplitude of 1-5 milliamps.
   Inventive Concept 86. The apparatus according to any one of Inventive Concepts 59, 63, and 71-73,
wherein the second SPG-stimulation site is outside a nasal cavity of the subject, and
wherein the second SPG-stimulation electrode is non-invasive and is configured to be non-invasively applied to the second SPG-stimulation site outside the nasal cavity.

There is also provided, in accordance with an Inventive Concept 87 of the present invention, a method including:
non-invasively applying a first non-invasive transcranial Direct Current Stimulation (tDCS) electrode to a first tDCS site of a subject on an external surface of skin of a temple of the subject, the subject identified as at risk of or suffering from dementia;
non-invasively applying a second non-invasive tDCS electrode to a non-cephalic second tDCS site of the subject on the external surface of the skin; and
activating circuitry to drive, between the first and the second non-invasive tDCS electrodes, direct current configured to promote amyloid beta clearance from a brain of the subject.
   Inventive Concept 88. The method according to Inventive Concept 87,
wherein the dementia is Alzheimer's disease, and
wherein non-invasively applying the first non-invasive tDCS electrode includes non-invasively applying the first non-invasive tDCS electrode to the first tDCS site of the subject identified as at risk of or suffering from Alzheimer's disease.
   Inventive Concept 89. The method according to Inventive Concept 87, wherein the first tDCS site is on the external surface of the skin of the temple overlying a temporal bone or a sphenoid bone of the subject.
   Inventive Concept 90. The method according to Inventive Concept 87, wherein the non-cephalic second tDCS site in on the external surface of the skin of a part of the subject selected from the group consisting of: a neck and a shoulder.
   Inventive Concept 91. The method according to Inventive Concept 90, wherein the non-cephalic second tDCS site is on the external surface of a nape of the subject.
   Inventive Concept 92. The method according to Inventive Concept 87, wherein activating the circuitry includes activating the circuitry to configure the first non-invasive tDCS electrode to be an anode and the second non-invasive tDCS electrode to be a cathode.
   Inventive Concept 93. The method according to Inventive Concept 87, wherein the first and the second tDCS sites are ipsilateral.
   Inventive Concept 94. The method according to Inventive Concept 87, wherein the first and the second tDCS sites are contralateral.
   Inventive Concept 95. The method according to Inventive Concept 87, wherein activating the circuitry includes activating the circuitry to drive the direct current at an amplitude of 1-5 milliamps.
   Inventive Concept 96. The method according to Inventive Concept 87, wherein activating the circuitry includes activating the circuitry to further cause vibration of at least one of the first and the second non-invasive tDCS electrodes at a frequency of 30-60 Hz.

There is further provided, in accordance with an Inventive Concept 97 of the present invention, apparatus including:
a first non-invasive transcranial Direct Current Stimulation (tDCS) electrode, configured to be applied to a first tDCS site of a subject on an external surface of skin of a temple of the subject;
a second non-invasive tDCS electrode, configured to be applied to a non-cephalic second tDCS site of the subject on the external surface of the skin; and
circuitry, which is configured to drive, between the first and the second non-invasive tDCS electrodes, direct current configured to promote amyloid beta clearance from a brain of the subject.
   Inventive Concept 98. The apparatus according to Inventive Concept 97, wherein the circuitry is configured to configure the first non-invasive tDCS electrode to be an anode and the second non-invasive tDCS electrode to be a cathode.
   Inventive Concept 99. The apparatus according to Inventive Concept 97, wherein the circuitry is configured to drive the direct current at an amplitude of 1-5 milliamps.
   Inventive Concept 100. The apparatus according to any one of Inventive Concepts 97-99, wherein the second non-invasive tDCS electrode is configured to be applied to the first tDCS site on the external surface of the skin of the temple overlying a temporal bone or a sphenoid bone of the subject.
   Inventive Concept 101. The apparatus according to any one of Inventive Concepts 97-99, wherein the second non-invasive tDCS electrode is configured to be applied to the non-cephalic second tDCS site on the external surface of the skin of a part of the subject selected from the group consisting of: a neck and a shoulder.
   Inventive Concept 102. The apparatus according to any one of Inventive Concepts 97-99, wherein the circuitry is configured to cause vibration of at least one of the first and the second non-invasive tDCS electrodes at a frequency of 30-60 Hz.

There is still further provided, in accordance with an Inventive Concept 103 of the present invention, a method including:
non-invasively applying first and second non-invasive transcranial Direct Current Stimulation (tDCS) electrodes to first and second tDCS sites of a subject on an external surface of skin of the subject, respectively, the subject identified as at risk of or suffering from dementia; and
activating circuitry to:
   drive, between the first and the second non-invasive tDCS electrodes, direct current configured to promote amyloid beta clearance from a brain of the subject, and
   cause vibration of at least one of the first and the second non-invasive tDCS electrodes at a frequency of 30-60 Hz.
      Inventive Concept 104. The method according to Inventive Concept 103,
wherein the dementia is Alzheimer's disease, and
wherein non-invasively applying the first and the second non-invasive tDCS electrode includes non-invasively applying the first and the second non-invasive tDCS electrodes to the first and the second tDCS sites, respectively, on the external surface of the skin of the subject identified as at risk of or suffering from Alzheimer's disease.
   Inventive Concept 105. The method according to Inventive Concept 103, wherein activating the circuitry includes activating the circuitry to drive the direct current at an amplitude of 1-5 milliamps.

There is additionally provided, in accordance with an Inventive Concept 106 of the present invention, apparatus including:
first and second non-invasive transcranial Direct Current Stimulation (tDCS) electrodes, configured to be applied to first and second tDCS sites of a subject on an external surface of skin of the subject, respectively;
circuitry, which is configured to:
   drive, between the first and the second non-invasive tDCS electrodes, direct current configured to promote amyloid beta clearance from a brain of the subject, and
   cause vibration of at least one of the first and the second non-invasive tDCS electrodes at a frequency of 30-60 Hz.
      Inventive Concept 107. The apparatus according to Inventive Concept 106, wherein the circuitry is configured to drive the direct current at an amplitude of 1-5 milliamps.

There is yet additionally provided, in accordance with an Inventive Concept 108 of the present invention, a method including:
stimulation electrode to a first SPG-stimulation site of a subject identified as at risk of or suffering from dementia;
applying a second SPG-stimulation electrode to a second SPG-stimulation site of the subject;
non-invasively applying first and second non-invasive transcranial Direct Current Stimulation (tDCS) electrodes to an external surface of skin at first and second tDCS sites, respectively; and
activating circuitry to:
   drive, between the first non-invasive SPG-stimulation electrode and the second SPG-stimulation electrode, alternating current configured to increase cerebral blood flow (CBF) of the subject by stimulating an SPG of the subject,
   drive, between the first and the second non-invasive tDCS electrodes, direct current configured to promote amyloid beta clearance from a brain of the subject, and
   apply to the subject gamma band entrainment stimulation that modulates oscillatory brain activity of the subject.
      Inventive Concept 109. The method according to Inventive Concept 108,
wherein the dementia is Alzheimer's disease, and
wherein non-invasively applying the first non-invasive SPG-stimulation electrode includes non-invasively applying the first non-invasive SPG-stimulation electrode to the first SPG-stimulation site of the subject identified as at risk of or suffering from Alzheimer's disease.
   Inventive Concept 110. The method according to Inventive Concept 108, wherein applying the second SPG-stimulation electrode includes non-invasively applying the second SPG-stimulation electrode to the second SPG-stimulation site.
   Inventive Concept 111. The method according to Inventive Concept 110,
wherein the first and the second SPG-stimulation sites are on a hard palate in a vicinity of a greater palatine nerve, and
wherein applying the first and the second SPG-stimulation electrodes includes non-invasively applying the first and the second SPG-stimulation electrodes to the first and the second SPG-stimulation sites within an oral cavity of the subject.
   Inventive Concept 112. The method according to Inventive Concept 108,
wherein the first SPG-stimulation site is on the external surface of the skin of the subject, and
wherein non-invasively applying the first non-invasive SPG-stimulation electrode to the first SPG-stimulation site includes non-invasively applying the first non-invasive SPG-stimulation electrode to the first SPG-stimulation site on the external surface of the skin of the subject.
   Inventive Concept 113. The method according to Inventive Concept 112,
wherein a headphone (a) is coupled to the first non-invasive SPG-stimulation electrode and the first and the second non-invasive tDCS electrodes, and (b) includes one or two earphones, which include one or more speakers,
wherein non-invasively applying the first non-invasive SPG-stimulation electrode and the first and the second non-invasive tDCS electrodes includes coupling the headphone to a head of the subject such that the headphone non-invasively applies:
   the first non-invasive SPG-stimulation electrode to the first SPG-stimulation site, and
   the first and the second non-invasive tDCS electrodes to the external surface of the skin at the first and the second tDCS sites, respectively,
wherein coupling the headphone to the head includes coupling the headphone to the head such that the headphone positions the one or two earphones over or in one or two respective ears of the subject, and
wherein activating the circuitry includes activating the circuitry to drive the one or more speakers of the one or two earphones to apply, to the one or two ears, auditory gamma band entrainment stimulation that modulates the oscillatory brain activity.
   Inventive Concept 114. The method according to Inventive Concept 113, wherein activating the circuitry includes activating the circuitry to drive the one or more speakers of the one or two earphones to apply the auditory gamma band entrainment stimulation at a frequency of 30-60 Hz.
   Inventive Concept 115. The method according to Inventive Concept 108, wherein activating the circuitry includes activating the circuitry to drive the direct current at an amplitude of 1-5 milliamps.
   Inventive Concept 116. The method according to Inventive Concept 108, wherein activating the circuitry includes activating the circuitry to drive the alternating current in pulses having a pulse width of 50-5,000 microseconds and a frequency of 10-60 Hz, e.g., 10-40 Hz.
   Inventive Concept 117. The method according to Inventive Concept 108, wherein activating the circuitry includes activating the circuitry to drive the alternating current at a frequency of 10-60 Hz, e.g., 10-40 Hz.
   Inventive Concept 118. The method according to Inventive Concept 108, wherein activating the circuitry includes activating the circuitry to drive the alternating current in pulses having a pulse width of 50-5,000 microseconds.
   Inventive Concept 119. The method according to Inventive Concept 108, wherein activating the circuitry includes activating the circuitry to further cause vibration of at least one of the first and the second non-invasive tDCS electrodes at a frequency of 30-60 Hz.
   Inventive Concept 120. The method according to Inventive Concept 108, wherein activating the circuitry includes activating the circuitry to apply, to one or two ears of the subject, auditory gamma band entrainment stimulation that modulates the oscillatory brain activity.
   Inventive Concept 121. The method according to Inventive Concept 108, wherein activating the circuitry includes activating the circuitry to apply somatosensory gamma band entrainment stimulation that modulates the oscillatory brain activity.
   Inventive Concept 122. The method according to Inventive Concept 108, wherein activating the circuitry includes activating the circuitry to apply transcranial Direct Alternating Stimulation (tACS) gamma band entrainment stimulation that modulates the oscillatory brain activity.

There is also provided, in accordance with an Inventive Concept 123 of the present invention, apparatus including:
a first non-invasive sphenopalatine ganglion (SPG)-stimulation electrode, configured to be applied to a first SPG-stimulation site of a subject;
a second SPG-stimulation electrode, configured to be applied to a second SPG-stimulation site of the subject;
first and second non-invasive transcranial Direct Current Stimulation (tDCS) electrodes, configured to be applied to an external surface of skin at first and second tDCS sites, respectively; and
circuitry, which is configured to:
   drive, between the first non-invasive SPG-stimulation electrode and the second SPG-stimulation electrode, alternating current configured to increase cerebral blood flow (CBF) of the subject by stimulating an SPG of the subject,
   drive, between the first and the second non-invasive tDCS electrodes, direct current configured to promote amyloid beta clearance from a brain of the subject, and
   apply to the subject gamma band entrainment stimulation that modulates oscillatory brain activity of the subject.
      Inventive Concept 124. The apparatus according to Inventive Concept 123, wherein the circuitry is configured to drive the direct current at an amplitude of 1-5 milliamps.
      Inventive Concept 125. The apparatus according to Inventive Concept 123, wherein the circuitry is configured to drive the alternating current in pulses having a pulse width of 50-5,000 microseconds and a frequency of 10-60 Hz, e.g., 10-40 Hz.
      Inventive Concept 126. The apparatus according to Inventive Concept 123, wherein the circuitry is configured to drive the alternating current at a frequency of 10-60 Hz, e.g., 10-40 Hz.
      Inventive Concept 127. The apparatus according to Inventive Concept 123, wherein the circuitry is configured to drive the alternating current in pulses having a pulse width of 50-5,000 microseconds.
      Inventive Concept 128. The apparatus according to Inventive Concept 123, wherein the circuitry is configured to cause vibration of at least one of the first and the second non-invasive tDCS electrodes at a frequency of 30-60 Hz.
      Inventive Concept 129. The apparatus according to any one of Inventive Concepts 123-128, wherein the second SPG-stimulation electrode includes a second non-invasive SPG-stimulation electrode.
      Inventive Concept 130. The apparatus according to Inventive Concept 129,
wherein the first and the second SPG-stimulation sites are on a hard palate in a vicinity of a greater palatine nerve, and
wherein the first and the second SPG-stimulation electrodes are configured to be applied to the first and the second SPG-stimulation sites within an oral cavity of the subject.
   Inventive Concept 131. The apparatus according to any one of Inventive Concepts 123-128,
wherein the first SPG-stimulation site is on the external surface of the skin of the subject, and
wherein the first non-invasive SPG-stimulation electrode is configured to be applied to the first SPG-stimulation site on the external surface of the skin of the subject.
   Inventive Concept 132. The apparatus according to Inventive Concept 129,
further including a headphone, which include one or two earphones including one or more speakers,
wherein the headphone is coupled to the first non-invasive SPG-stimulation electrode and the first and the second non-invasive tDCS electrodes,
wherein the headphone is configured, when worn on a head of the subject, to:
   non-invasively apply (a) the first non-invasive SPG-stimulation electrode to the first SPG-stimulation site, and (b) the first and the second non-invasive tDCS electrodes to the external surface of the skin at the first and the second tDCS sites, respectively, and
   position the one or two earphones over or in one or two respective ears of the subject, and
wherein the circuitry is configured to drive the one or more speakers of the one or two earphones to apply, to the one or two ears, auditory gamma band entrainment stimulation that modulates the oscillatory brain activity.
   Inventive Concept 133. The apparatus according to Inventive Concept 132, wherein the circuitry is configured to drive the one or more speakers of the one or two earphones to apply the auditory gamma band entrainment stimulation at a frequency of 30-60 Hz.
   Inventive Concept 134. The apparatus according to any one of Inventive Concepts 123-128,
further including one or more speakers, configured to applied to one or two ears of the subject, and
wherein the circuitry is configured to drive the one or more speakers to apply, to the one or two ears of the subject, auditory gamma band entrainment stimulation that modulates the oscillatory brain activity.
   Inventive Concept 135. The apparatus according to any one of Inventive Concepts 123-128,
further including one or more somatosensory stimulators, and
wherein the circuitry is configured to drive the one or more somatosensory stimulators to apply somatosensory gamma band entrainment stimulation that modulates the oscillatory brain activity.
   Inventive Concept 136. The apparatus according to any one of Inventive Concepts 123-128,
further including two or more non-invasive transcranial Direct Alternating Stimulation (tACS) electrodes, and
wherein the circuitry is configured to drive the two or more non-invasive tACS electrodes to apply tACS gamma band entrainment stimulation that modulates the oscillatory brain activity.
   Inventive Concept 137. The apparatus according to any one of Inventive Concepts 123-128,
further including one or more non-invasive light sources, and
wherein the circuitry is configured to drive the one or more non-invasive light sources to apply non-invasive light flicker gamma band entrainment stimulation that modulates the oscillatory brain activity.

There is further provided, in accordance with an Inventive Concept 138 of the present invention, a method comprising:
inserting a neurostimulator into a face of a subject and advancing the neurostimulator between a mandibular notch and a zygomatic process of a temporal bone toward a sphenopalatine ganglion (SPG), the subject identified as at risk of or suffering from dementia; and
increasing cerebral blood flow (CBF) of the subject by activating the neurostimulator to drive, between first and second SPG-stimulation electrodes of the neurostimulator, alternating current configured to stimulate the SPG.
   Inventive Concept 139. The method according to Inventive Concept 138,
wherein the dementia is Alzheimer's disease, and
wherein inserting the neurostimulator comprises inserting the neurostimulator into the face of the subject identified as at risk of or suffering from Alzheimer's disease.
   Inventive Concept 140. The method according to Inventive Concept 138, wherein inserting the neurostimulator comprises injecting the neurostimulator through skin of the face.
   Inventive Concept 141. The method according to Inventive Concept 138, wherein activating the neurostimulator comprises activating the neurostimulator to drive the alternating current in pulses having a pulse width of 50-5,000 microseconds and a frequency of 10-60 Hz.
   Inventive Concept 142. The method according to Inventive Concept 138, wherein activating the neurostimulator comprises activating the neurostimulator to drive the alternating current at a frequency of 10-60 Hz.
   Inventive Concept 143. The method according to Inventive Concept 138, wherein activating the neurostimulator comprises activating the neurostimulator to drive the alternating current in pulses having a pulse width of 50-5,000 microseconds.
   Inventive Concept 144. The method according to Inventive Concept 138,
wherein the neurostimulator comprises an antenna coil, and
wherein activating the neurostimulator comprises wirelessly transmitting energy to the antenna coil from outside the face.
   Inventive Concept 145. The method according to Inventive Concept 144,
wherein the antenna coil is disposed closer to a first end of the neurostimulator than to a second end opposite the first end, and
wherein inserting the neurostimulator comprises inserting the neurostimulator into the face such that the first end of the neurostimulator is more superficial than the second end.
   Inventive Concept 146. The method according to Inventive Concept 144, wherein wirelessly transmitting the energy comprises:
coupling a head support to a head of the subject, the head support including circuitry; and
activating the circuitry to drive a wireless transmitter to wirelessly transmit the energy to the antenna coil of the neurostimulator.
   Inventive Concept 147. The method according to Inventive Concept 146, wherein the head support is configured to apply the wireless transmitter to an external surface of skin on the face of the subject.
   Inventive Concept 148. The method according to Inventive Concept 146, wherein the head support comprises a headphone, which comprises one or two earphones, and wherein the wireless transmitter is disposed within one of the one or two earphones.
   Inventive Concept 149. The method according to Inventive Concept 146,
wherein the head support comprises a headphone, which comprises one or two earphones, which comprise one or more speakers,
wherein coupling the head support to the head comprises coupling the headphone to the head such that the headphone positions the one or two earphones over or in one or two respective ears of the subject, and
wherein activating the circuitry comprises activating the circuitry to drive the one or more speakers of the one or two earphones to apply, to the one or two ears, auditory stimulation that modulates oscillatory brain activity of the subject.
   Inventive Concept 150. The method according to Inventive Concept 149, wherein activating the circuitry comprises activating the circuitry to drive the one or more speakers of the one or two earphones to apply the auditory stimulation at a frequency of 30-60 Hz.
   Inventive Concept 151. The method according to Inventive Concept 146,
wherein the head support is coupled to first and second non-invasive transcranial Direct Current Stimulation (tDCS) electrodes,
wherein coupling the head support to the head comprises coupling the head support to the head such that the head support applies:
   the first non-invasive tDCS electrode to an external surface of the skin at a first tDCS site on a temple of the subject, and
   the second non-invasive tDCS electrode to the external surface of the skin at a non-cephalic second tDCS site, and
   wherein activating the circuitry comprises activating the circuitry to drive, between the first and the second non-invasive tDCS electrodes, direct current configured to promote amyloid beta clearance from a brain of the subject.
      Inventive Concept 152. The method according to Inventive Concept 151, wherein coupling the head support to the head comprises coupling the head support to the head such that the head support applies the non-cephalic second non-invasive tDCS electrode to the external surface of the skin on a part of the subject selected from the group consisting of: a neck and a shoulder.
      Inventive Concept 153. The method according to Inventive Concept 152, wherein coupling the head support to the head comprises coupling the head support to the head such that the head support applies the non-cephalic second non-invasive tDCS electrode to the external surface of a nape of the subject.
      Inventive Concept 154. The method according to Inventive Concept 151, wherein activating the circuitry comprises activating the circuitry to drive the direct current at an amplitude of 1-5 milliamps.
      Inventive Concept 155. The method according to Inventive Concept 138, further comprising:
non-invasively applying first and second non-invasive transcranial Direct Current Stimulation (tDCS) electrodes to an external surface of skin at first and second tDCS sites, respectively; and
activating circuitry to:
   activating the neurostimulator to drive the alternating current between the first and second SPG-stimulation electrodes,
   drive, between the first and the second non-invasive tDCS electrodes, direct current configured to promote amyloid beta clearance from a brain of the subject, and
   apply to the subject gamma band entrainment stimulation that modulates oscillatory brain activity of the subject.

There is still further provided, in accordance with an Inventive Concept 156 of the present invention, apparatus comprising:
a neurostimulator, configured to be inserted into a face of a subject and advanced between a mandibular notch and a zygomatic process of a temporal bone toward a sphenopalatine ganglion (SPG), the neurostimulator comprising first and second SPG-stimulation electrodes;
first and second non-invasive transcranial Direct Current Stimulation (tDCS) electrodes, configured to be applied to an external surface of skin at first and second tDCS sites, respectively; and
circuitry, which is configured to:
   activate the neurostimulator to drive, between the first and the second SPG-stimulation electrodes, alternating current configured to increase cerebral blood flow (CBF) of the subject by stimulating the SPG,
   drive, between the first and the second non-invasive tDCS electrodes, direct current configured to promote amyloid beta clearance from a brain of the subject, and
   apply to the subject gamma band entrainment stimulation that modulates oscillatory brain activity of the subject.
      Inventive Concept 157. The apparatus according to Inventive Concept 156, wherein the circuitry is configured to drive the direct current at an amplitude of 1-5 milliamps.
      Inventive Concept 158. The apparatus according to Inventive Concept 156, wherein the circuitry is configured to activate the neurostimulator to drive the alternating current in pulses having a pulse width of 50-5,000 microseconds and a frequency of 10-60 Hz.
      Inventive Concept 159. The apparatus according to Inventive Concept 156, wherein the circuitry is configured to activate the neurostimulator to drive the alternating current at a frequency of 10-60 Hz.
      Inventive Concept 160. The apparatus according to Inventive Concept 156, wherein the circuitry is configured to activate the neurostimulator to drive the alternating current in pulses having a pulse width of 50-5,000 microseconds.
      Inventive Concept 161. The apparatus according to Inventive Concept 156, wherein the circuitry is configured to cause vibration of at least one of the first and the second non-invasive tDCS electrodes at a frequency of 30-60 Hz.
      Inventive Concept 162. The apparatus according to any one of Inventive Concepts 156-161, further comprising a wireless transmitter,
wherein the neurostimulator comprises an antenna coil,
wherein the apparatus further comprises a head support, which is configured to be coupled to a head of the subject, and which comprises circuitry that is configured to drive the wireless transmitter to wirelessly transmit energy to the antenna coil of the neurostimulator, and
wherein the first and the second non-invasive transcranial tDCS electrodes are coupled to the head support.
   Inventive Concept 163. The apparatus according to Inventive Concept 162, wherein the head support is configured to apply the wireless transmitter to an external surface of the skin on the face of the subject.
   Inventive Concept 164. The apparatus according to Inventive Concept 162, wherein the head support comprises a headphone, which comprises one or two earphones, and wherein the wireless transmitter is disposed within one of the one or two earphones.
   Inventive Concept 165. The apparatus according to Inventive Concept 162, wherein the head support is configured to apply:
the first non-invasive tDCS electrode to the external surface of the skin at a first tDCS site on a temple of the subject, and
the second non-invasive tDCS electrode to the external surface of the skin at a non-cephalic second tDCS site.
   Inventive Concept 166. The apparatus according to Inventive Concept 162,
wherein the head support comprises a headphone, which (a) comprises one or two earphones, which comprise one or more speakers, and (b) is configured to position the one or two earphones over or in one or two respective ears of the subject, and
wherein the circuitry is configured to drive the one or more speakers of the one or two earphones to apply, to the one or two ears, the gamma band entrainment stimulation as auditory stimulation that modulates oscillatory brain activity of the subject.

The present invention will be more fully understood from the following detailed description of embodiments thereof, taken together with the drawings, in which:

### BRIEF DESCRIPTION OF THE DRAWINGS

FIGS. 1A-D are schematic illustrations of respective configurations of a system for treating or preventing dementia, such as Alzheimer's disease, in accordance with respective applications of the present invention;

FIGS. 2A-B are schematic illustrations of the application of alternating current between a first non-invasive SPG-stimulation electrode and a second SPG-stimulation electrode, in accordance with an application of the present invention;

FIGS. 3A-F are schematic illustrations of the application of alternating current between a first non-invasive SPG-stimulation electrode and a second SPG-stimulation electrode, in accordance with respective applications of the present invention;

FIGS. 4A-E are schematic illustrations of respective oral supports, in accordance with respective applications of the present invention;

FIG. 5 is a schematic illustration of the insertion of a wire into a greater palatine canal, in accordance with an application of the present invention;

FIGS. 6A-B are schematic illustrations of application of first and second non-invasive tDCS electrodes, in accordance with respective applications of the present invention;

FIGS. 7A-B are schematic illustrations of direct current paths between the first and the second non-invasive tDCS electrodes of FIGS. 6A-B, in accordance with respective applications of the present invention;

FIG. 8 is a schematic illustration of direct current paths between two sets of first and second non-invasive tDCS electrodes, respectively, in accordance with an application of the present invention;

FIG. 9 is a schematic illustration of a neurostimulation system comprising a neurostimulator, in accordance with an application of the present invention;

FIG. 10 is a schematic illustration of a method of implanting the neurostimulator of FIG. 9, in accordance with an application of the present invention; and

FIGS. 11A-B are schematic illustrations showing the neurostimulator of FIG. 9 implanted in the face of the subject, in accordance with an application of the present invention.

### DETAILED DESCRIPTION OF APPLICATIONS

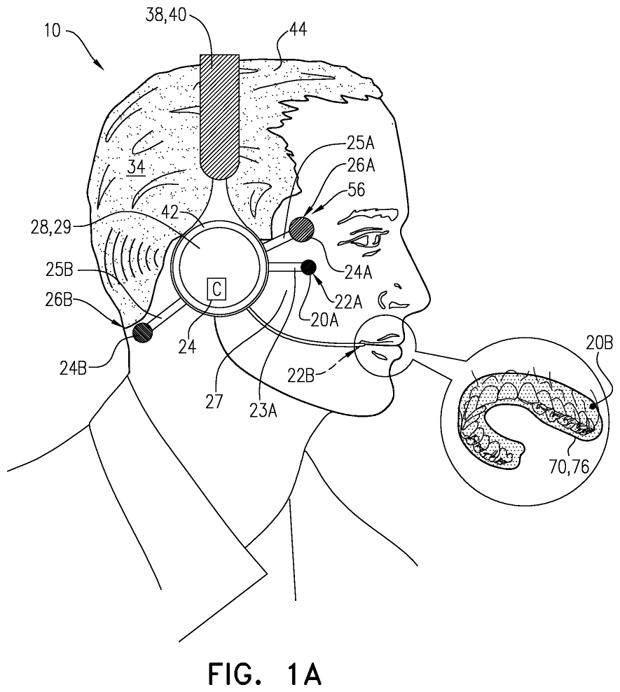

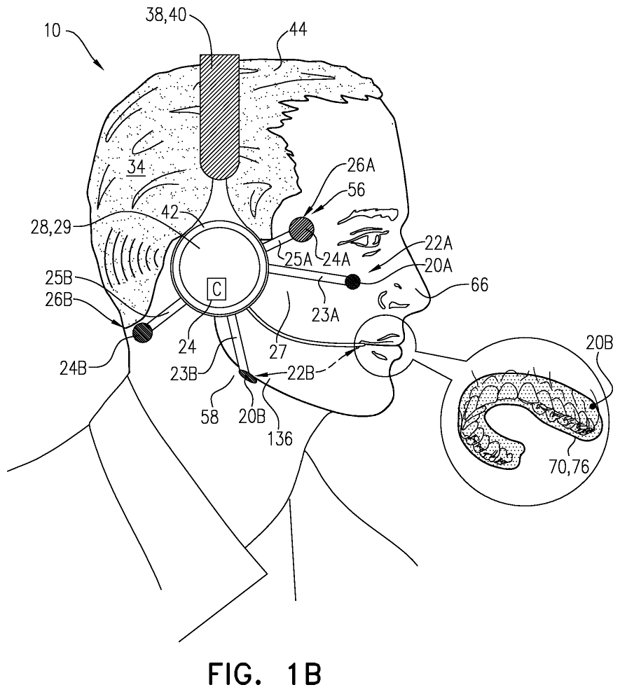

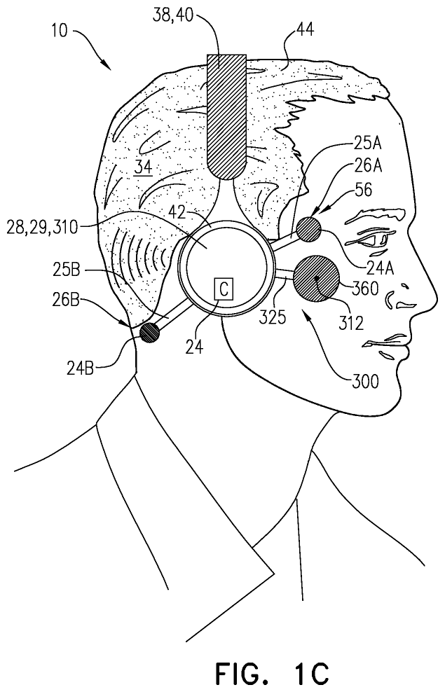

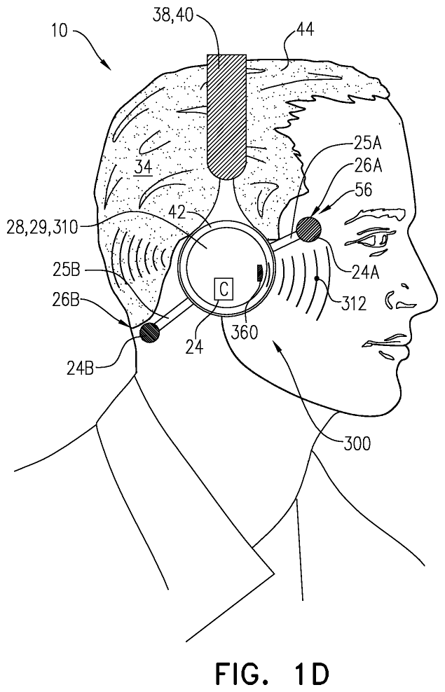

FIGS. 1A-D are schematic illustration of respective configurations of a system 10 for treating or preventing dementia, such as Alzheimer's disease, in accordance with respective applications of the present invention. System 10 may be used for treating early- or late-stage dementia, e.g., Alzheimer's, patients or preventing dementia, e.g., Alzheimer's disease. As used in the present application, including in the claims and Inventive Concepts, “preventing” dementia, e.g., Alzheimer's disease, means reducing the risk of dementia, e.g., Alzheimer's disease, in pre-clinical stage patients.

System 10 comprises one or more of the following elements:
first and second sphenopalatine ganglion (SPG)-stimulation electrodes;
first and second non-invasive transcranial Direct Current Stimulation (tDCS) electrodes 24A and 24B, configured to be applied to the external surface of skin 27 of the subject at first and second tDCS sites 26A and 26B, respectively; and
circuitry 32.

Circuitry 32 is typically configured to perform one or more of the following:
drive, between the first and the second SPG-stimulation electrodes, alternating current (AC) configured to increase cerebral blood flow (CBF) of the subject (and, typically, promotes vasodilation) by stimulating an SPG of the subject,
drive, between first and second non-invasive tDCS electrodes 24A and 24B, direct current configured to promote microglial cell activity and/or amyloid beta clearance from a brain 34 of the subject, and
apply to the subject gamma band entrainment stimulation that modulates oscillatory brain activity of the subject.

For some applications, circuitry 32 is configured to simultaneously apply two or three of the above-mentioned types of stimulation. Alternatively, circuitry 32 is configured to apply two or three of the above-mentioned types of stimulation during different respective time periods.

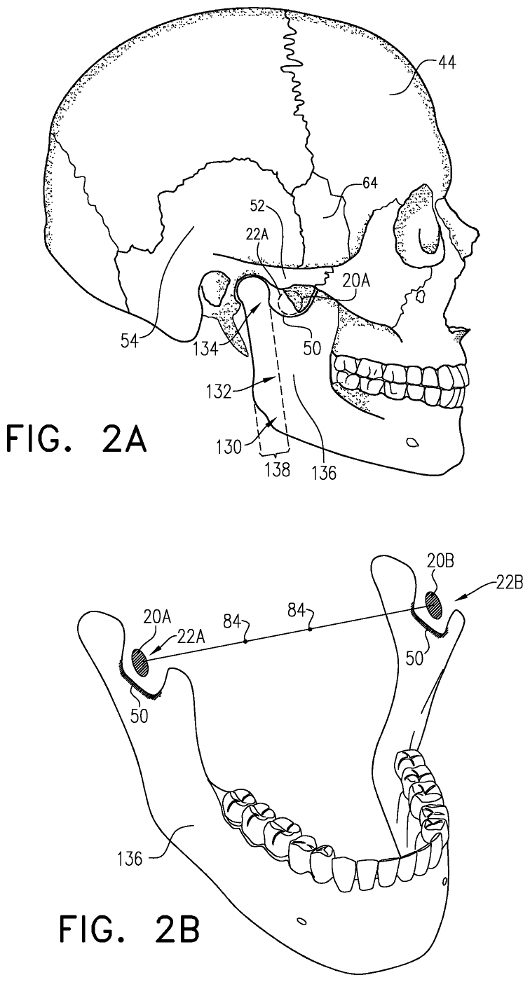

For some applications, such as shown in FIGS. 1A and 1B, as well as in FIGS. 2A-B, 3A-F, and 4A-D, described hereinbelow, the first and the second SPG-stimulation electrodes comprise:
a first non-invasive SPG-stimulation electrode 20A, configured to be applied to a first SPG-stimulation site 22A of a subject on an external surface of skin 27 of the subject; and
a second SPG-stimulation electrode 20B, configured to be applied to a second SPG-stimulation site 22B of the subject.

As used in the present application, including in the claims and Inventive Concepts, “non-invasive” means not penetrating tissue, and includes within its scope both application of electrodes on an external surface of the subject and on an internal surface of the subject, such as in the oral cavity.

Activation of circuitry 32 may be performed by the patient and/or remotely.

Although FIG. 1B shows two second SPG-stimulation electrodes 20B on a single side of the head (one on the skin and the other in the oral cavity), this is for illustrative purposes only; system 10 typically comprises only a single second SPG-stimulation electrode 20B on any given side of the head.

For some applications, system 10 comprises one or more gamma band entrainment stimulators 28, configured to be applied to the subject, and circuitry 32 is configured to drive the one or more gamma band entrainment stimulators 28 to apply the gamma band entrainment stimulation that modulates the oscillatory brain activity of the subject.

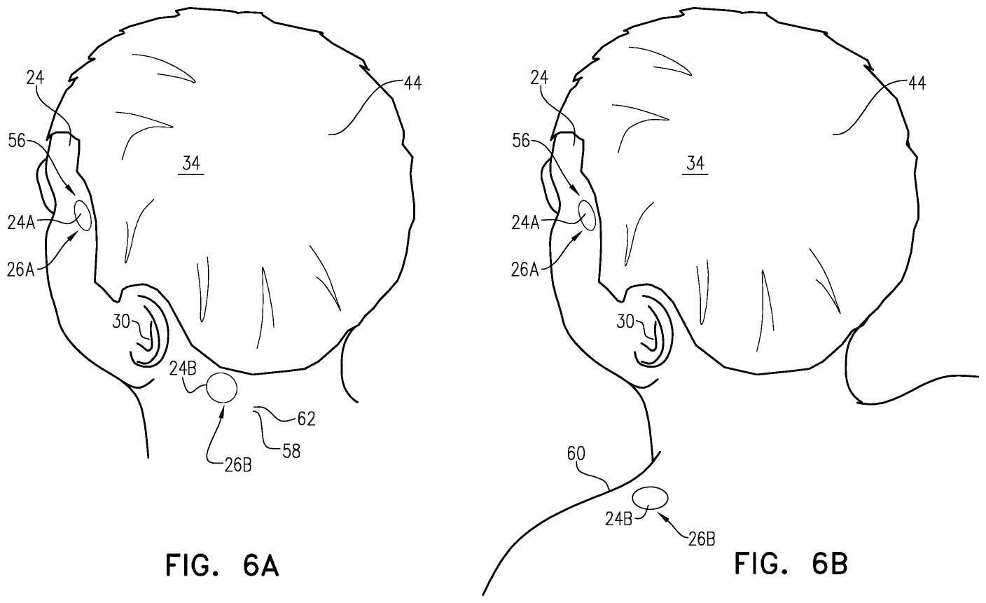

For some applications, the one or more gamma band entrainment stimulators 28 comprise one or more speakers 29, configured to applied to one or two ears 30 of the subject (labeled in FIGS. 6A-B). Circuitry 32 is configured to drive the one or more speakers 29 to apply, to the one or two ears 30, auditory gamma band entrainment stimulation that modulates the oscillatory brain activity. For some of these applications, system 10 further comprises a head support 38, i.e., a head mount for mounting one or more elements of system 10 on the head of the subject, such that the head supports the one or more elements. Head support 38 optionally comprises a headphone 40, which comprises one or two earphones 42 comprising the one or more speakers 29. Headphone 40 is configured to position the one or two earphones 42 over or in one or two respective ears 30 of the subject.

Optionally, the one or more gamma band entrainment stimulators 28 are configured to vibrate in order to apply and/or enhance the gamma band entrainment.

For some applications, such as shown in FIGS. 1A-B, headphone 40 is coupled to first non-invasive SPG-stimulation electrode 20A and first and second non-invasive tDCS electrodes 24A and 24B. Headphone 40 is configured, when worn on a head 44 of the subject, to:
non-invasively apply (a) first non-invasive SPG-stimulation electrode 20A to first SPG-stimulation site 22A, and, optionally, second non-invasive SPG-stimulation electrode 20B to second SPG-stimulation site 22B, and (b) first and second non-invasive tDCS electrodes 24A and 24B to the external surface of skin 27 at first and second tDCS sites 26A and 26B, respectively, and
position the one or two earphones 42 over or in one or two respective ears 30 of the subject.
   Circuitry 32 is configured to drive the one or more speakers 29 of the one or two earphones 42 to apply, to the one or two ears 30, the auditory gamma band entrainment stimulation that modulates oscillatory brain activity.

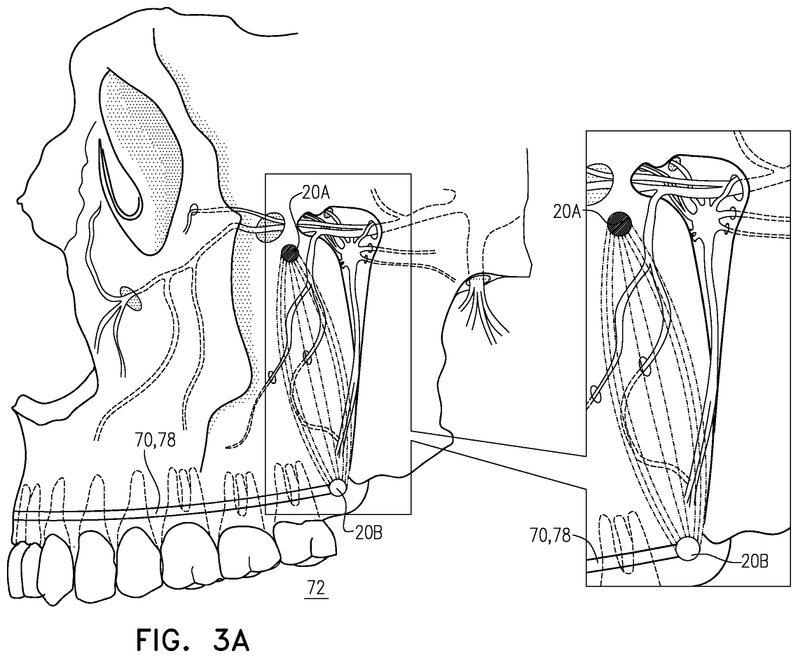

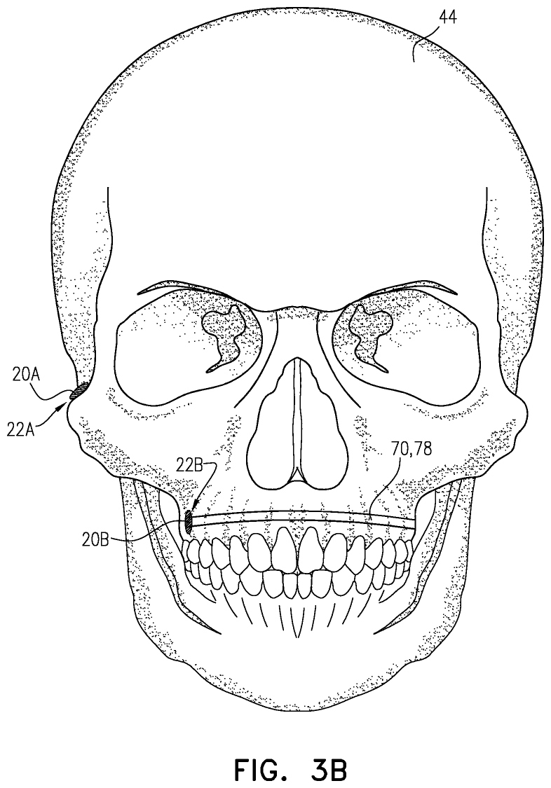

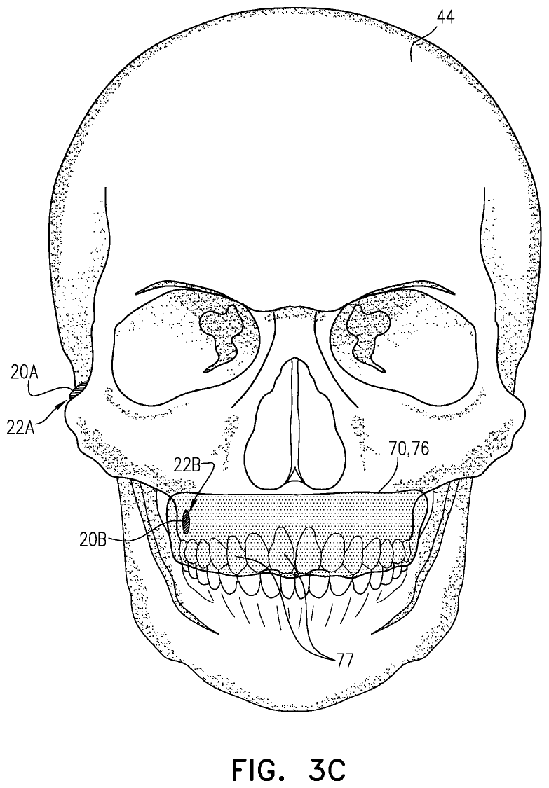

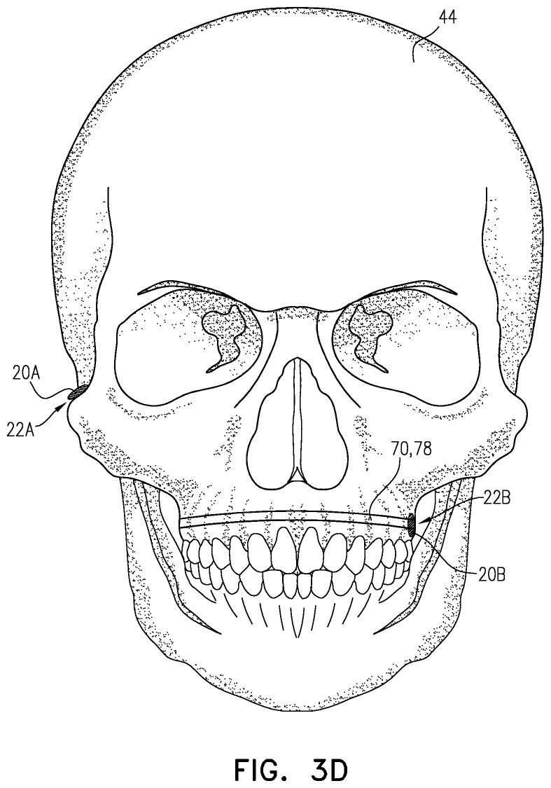

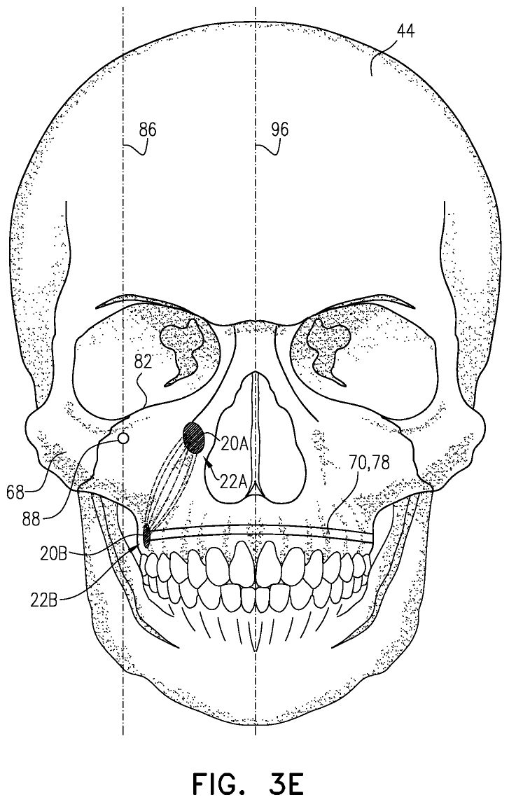

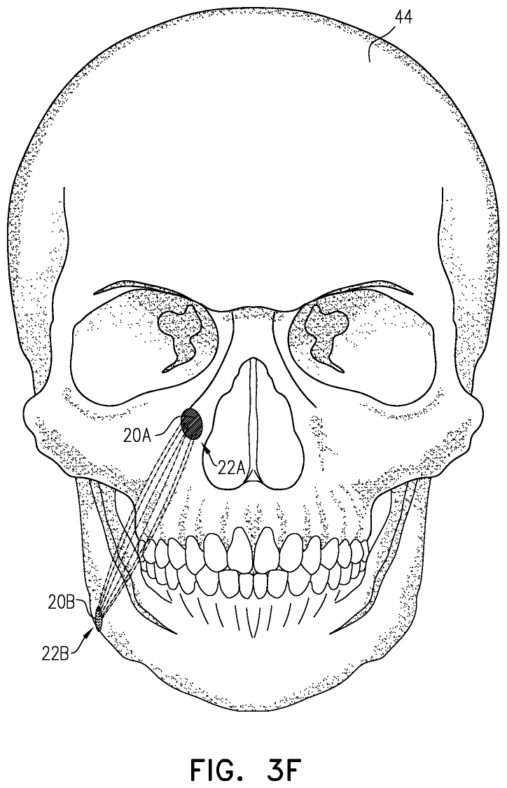

For some applications, such as shown in FIGS. 1A-B, headphone 40 comprises:
a first SPG electrode support arm 23A, to which first non-invasive SPG-stimulation electrode 20A is coupled;
first and second tDCS electrode support arms 25A and 25B, to which first and second non-invasive tDCS electrodes 24A and 24B are respectively coupled; and
optionally, in the configuration shown in FIG. 1B and described hereinbelow with reference to FIGS. 1B and 3E, a second SPG electrode support arm 23B, to which second non-invasive SPG-stimulation electrode 20B is coupled.

For some applications, the first and the second SPG-stimulation electrode comprise implantable (and thus invasive) first and second SPG-stimulation electrodes 320A and 320B, such as described hereinbelow with reference to FIGS. 1C-D, 9, 10, and 11A-B.

For some applications, headphone 40 is configured such that positions of one or more of the supports arms are adjustable with respect to the one or two earphones 42 (e.g., can swivel), in order to allow precise selection of the respective stimulation sites.

Typically, circuitry 32 comprises one or more microprocessors, memory, and/or programming instructions configured to carry out the functions of circuitry 32 described herein. The programming instructions may be implemented in hardware or software, or a combination of hardware and software. The elements of circuitry 32 may be implemented in one or more units, each having its own casing or housing, which may or may not be interconnected with one another.

### SPG Stimulation

For clarity of illustration, SPG stimulation is generally shown and described with respect to a single SPG 84 (with the exception of FIG. 1B). In any of the configurations described herein, the SPG stimulation may be applied to either a single SPG (the left or the right SPG) or both SPGs, in which case many of the elements of the system are duplicated for the left and right sides of the head.

As described above, for some applications, the first and the second SPG-stimulation electrodes comprise first and second non-invasive SPG-stimulation electrodes 20A and 20B. Reference is still made to FIGS. 1A-B and is additionally made to FIGS. 2A-B, which are schematic illustrations of the application of alternating current between first non-invasive SPG-stimulation electrode 20A and second SPG-stimulation electrode 20B, in accordance with an application of the present invention.

Reference is also made to FIGS. 3A-F, which are schematic illustrations of the application of alternating current between first non-invasive SPG-stimulation electrode 20A and second SPG-stimulation electrode 20B, in accordance with respective applications of the present invention. In some of these configurations, second SPG-stimulation electrode 20B is non-invasive, such as shown FIGS. 1A-B, 2A-B, and 3A-F. Although, for the sake of clarity, stimulation is shown applied to only a single side in FIGS. 3A-F, stimulation is generally applied bilaterally.

As described above, for some applications, circuitry 32 is configured to drive, between first non-invasive SPG-stimulation electrode 20A and second SPG-stimulation electrode 20B, alternating current configured to increase cerebral blood flow (CBF) of the subject by stimulating an SPG of the subject. This SPG stimulation promotes vasodilation and increased cerebral blood flow, and typically impacts the entire brain.

As mentioned above, head support 38 is coupled to first non-invasive SPG-stimulation electrode 20A. For some applications, such as shown in FIG. 1A (and in FIGS. 2A-B, and 3A-D, without showing head support 38), head support 38 is configured to non-invasively apply first non-invasive SPG-stimulation electrode 20A to first SPG-stimulation site 22A on the external surface of skin 27 between a mandibular notch 50 and a zygomatic process 52 of a temporal bone 54, when head support 38 is worn on head 44 of the subject. Optionally, first non-invasive SPG-stimulation electrode 20A is positioned within the slight depression of soft tissue of the face over the area between mandibular notch 50 and zygomatic process 52 of temporal bone 54, which may help properly position the electrode and/or hold the electrode properly positioned.

The current paths shown in some of the figures are highly schematic. In general, the inventors hypothesize that the current travels in multiple paths based on tissue resistance and thus pass through SPG 84. For example, the current generally encounters lower resistance when passing through soft tissue than through bone, and thus encounters lower resistance when passing through the area between mandibular notch 50 and zygomatic process 52 of temporal bone 54, or through greater palatine foramen 74.

In this configuration, head support 38 may or may not comprise headphone 40.

For some applications, second SPG-stimulation electrode 20B comprises a second non-invasive SPG-stimulation electrode 20B. For some of these applications, second non-invasive SPG-stimulation electrode 20B is applied outside a nasal cavity of the subject, such as shown in all of the figures that show second non-invasive SPG-stimulation electrode 20B.

For some of these applications in which second SPG-stimulation electrode 20B is non-invasive, mandibular notch 50 and zygomatic process 52 of temporal bone 54 are a left mandibular notch 50 and a left zygomatic process 52 of a left temporal bone 54, respectively. FIG. 2B shows a portion of the anatomy of this configuration, including a highly schematic labeling of the approximate locations of the left and right SPGs 84. FIG. 2B also highly schematically shows first and second non-invasive SPG-stimulation electrodes 20A and 20B, at their approximate respective first and second SPG-stimulation sites 22A and 22B.

For some applications, head support 38 is (a) coupled to first and second non-invasive SPG-stimulation electrodes 20A and 20B, and (b) configured to non-invasively apply, when head support 38 is worn on head 44 of the subject:
first non-invasive SPG-stimulation electrode 20A to first SPG-stimulation site 22A on the external surface of skin 27 between left mandibular notch 50 and left zygomatic process 52 of left temporal bone 54, and
second non-invasive SPG-stimulation electrode 20B to second SPG-stimulation site 22B on the external surface of skin 27 between a right mandibular notch 50 and a right zygomatic process 52 of a right temporal bone 54.

Driving of alternating current by circuitry 32 between first and second non-invasive SPG-stimulation electrodes 20A and 20B at these respective locations generally causes the alternating current to pass through both the left SPG 84 and the right SPG 84 of the subject.

For other applications, such as shown in FIG. 1B and partially labeled in FIG. 3E, head support 38 is configured to non-invasively apply first non-invasive SPG-stimulation electrode 20A to first SPG-stimulation site 22A on the external surface of skin 27 lateral to a nose 66, medial to a zygomatic bone 68 (labeled in FIG. 3E), and inferior to an infraorbital margin 82 (labeled in FIG. 3E), when head support 38 is worn on head 44 of the subject. (As used in this context, “lateral” is used in the anatomical sense of opposite to the “medial.” The phrase “lateral to the nose” means alongside the nose.) Optionally, first SPG-stimulation site 22A is medial to a plane 86 that is parallel to a sagittal plane 96 and includes an infraorbital foramen 88 (all labeled in FIG. 3E).

In this configuration, head support 38 may or may not comprise headphone 40.

As described above, for some applications, second SPG-stimulation electrode 20B comprises a second non-invasive SPG-stimulation electrode 20B. For some of these applications, such as shown in FIG. 1B, partially labeled in FIG. 2A, and shown in FIG. 3F (without showing head support 38), head support 38 is configured to non-invasively apply second non-invasive SPG-stimulation electrode 20B to second SPG-stimulation site 22B on the external surface of skin 27:
over an angle 130, a ramus 132, or a neck 134 of a mandible 136 (for example, over a posterior third 138 of angle 130, ramus 132, or neck 134 of mandible 136, or
on a neck 58 of the subject, typically near mandible 136.
   Driving of alternating current by circuitry 32 between first and second non-invasive SPG-stimulation electrodes 20A and 20B at these respective locations generally causes the alternating current to pass through an SPG 84 (the left or right SPG, depending on the side of the head to which the alternating current is applied).

Reference is still made to FIGS. 1A-B and 2A-B. In some applications of the present invention, a method is provided that comprises:
non-invasively applying first non-invasive SPG-stimulation electrode 20A to first SPG-stimulation site 22A on the external surface of skin 27, the subject identified as at risk of or suffering from dementia, e.g., Alzheimer's disease, the site selected from the group of sites consisting of: (a) a site between mandibular notch 50 and zygomatic process 52 of temporal bone 54, and (b) a site lateral to nose 66, medial to zygomatic bone 68, and inferior to infraorbital margin 82;
applying second SPG-stimulation electrode 20B to second SPG-stimulation site 22B; and
increasing cerebral blood flow (CBF) of the subject by activating circuitry 32 to drive, between first non-invasive SPG-stimulation electrode 20A and second SPG-stimulation electrode 20B, alternating current configured to stimulate the SPG of the subject.

For some applications, second SPG-stimulation electrode 20B is non-invasively applied to second SPG-stimulation site 22B.

For some of these applications:
mandibular notch 50 and zygomatic process 52 of temporal bone 54 are a left mandibular notch 50 and a left zygomatic process 52 of a temporal bone 54, respectively, and
second SPG-stimulation site 22B is on the external surface of skin 27 between a right mandibular notch 50 and a right zygomatic process 52 of a right temporal bone 54.

Reference is still made to FIGS. 1A-B and 2A-B. In some application of the present invention, a method is provided that comprises:
non-invasively applying first non-invasive SPG-stimulation electrode 20A to SPG-stimulation site 22A on the external surface of skin 27 of a subject identified as at risk of or suffering from dementia, e.g., Alzheimer's disease;
applying second SPG-stimulation electrode 20B to second SPG-stimulation site 22B of the subject;
non-invasively applying first and second non-invasive transcranial Direct Current Stimulation (tDCS) 24A and 24B electrodes to the external surface of skin 27 at first and second tDCS sites 26A and 26B, respectively; and
activating circuitry 32 to:
   drive, between first non-invasive SPG-stimulation electrode 20A and second SPG-stimulation electrode 20B, alternating current configured to increase cerebral blood flow (CBF) of the subject by stimulating an SPG of the subject,
   drive, between first and second non-invasive tDCS electrodes 24A and 24B, direct current configured to promote amyloid beta clearance from a brain of the subject, and
   apply to the subject gamma band entrainment stimulation that modulates oscillatory brain activity of the subject.

Reference is made to FIGS. 1A-B. In an application of the present invention, a method is provided that comprises non-invasively applying:
first non-invasive SPG-stimulation electrode 20A to the external surface of skin 27 of head 44 at first SPG-stimulation site 22A, the subject identified as at risk of or suffering from dementia, e.g., Alzheimer's disease, and
second non-invasive SPG-stimulation electrode 20B to the external surface of skin 27 at second SPG-stimulation site 22B selected from the group of sites consisting of: a side on head 44, a site on neck 58, and a site on shoulder 60.

First and second non-invasive SPG-stimulation electrodes 20A and 20B are applied such that a line between the first and the second SPG-stimulation sites passes within 1 cm of SPG 84.

CBF of the subject is increased by activating circuitry 32 to drive, between first and second non-invasive SPG-stimulation electrode 20A and 20B, alternating current configured to stimulate the SPG.

For some applications, first non-invasive SPG-stimulation electrode 20A is non-invasively applied to the external surface of skin 27 of a face of head 44 at first SPG-stimulation site 22A.

Alternatively or additionally, for some applications, second non-invasive SPG-stimulation electrode 20B is non-invasively applied to the external surface of skin 27 of the face of head 44 at second SPG-stimulation site 22B.

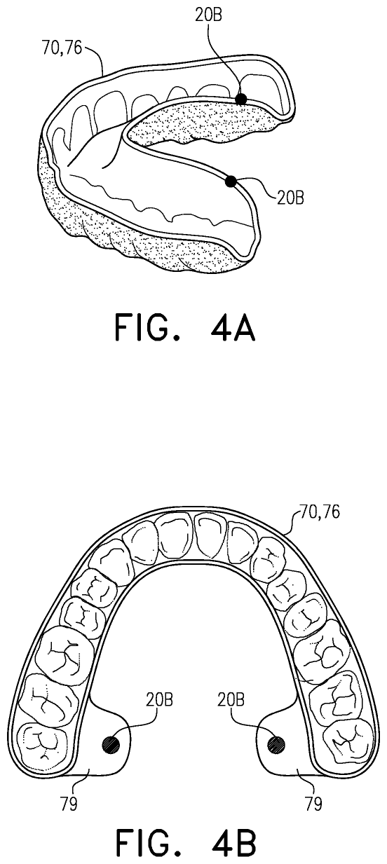

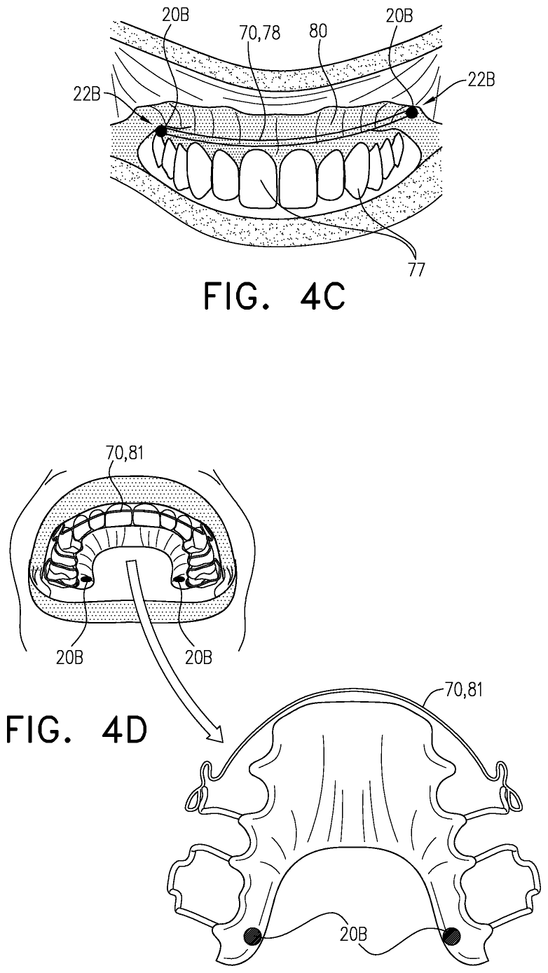

Reference is made to FIGS. 1A-B and FIGS. 3A-E, and is additionally made to FIGS. 4A-D, which are schematic illustrations of respective oral supports 70, in accordance with respective applications of the present invention.

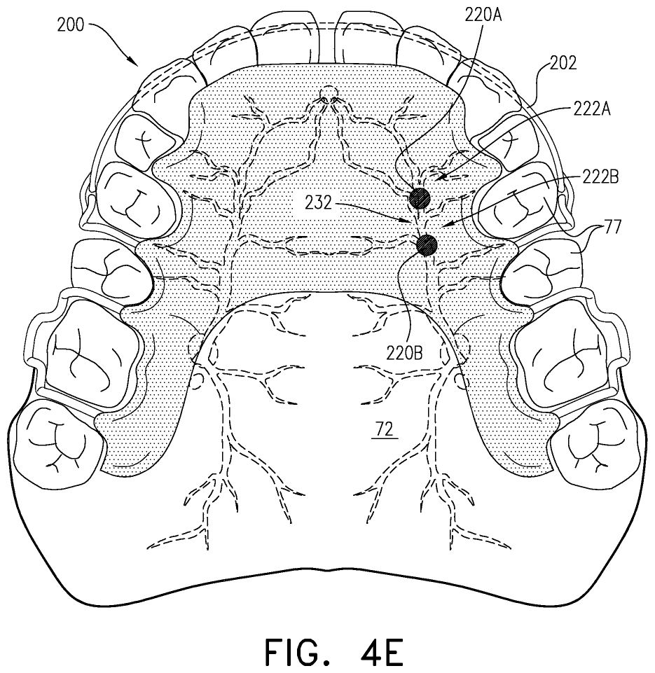

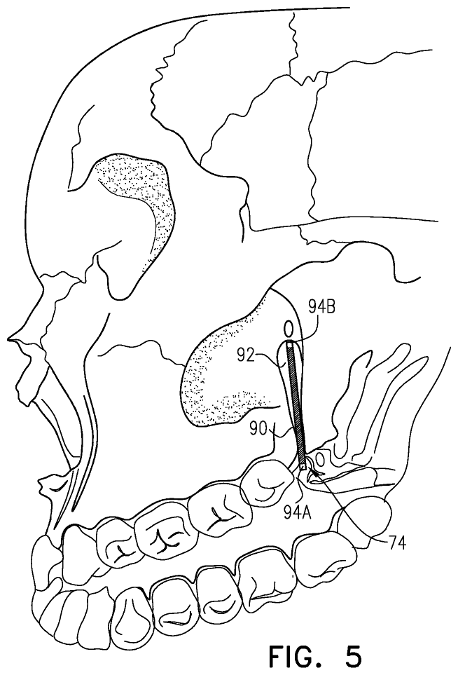

As mentioned above, for some applications, second SPG-stimulation electrode 20B comprises a second non-invasive SPG-stimulation electrode 20B. For some of these applications, such as shown in FIGS. 1A-B and 3A-E, second SPG-stimulation site 22B is within an oral cavity 72 (labeled in FIG. 3A and FIG. 5, described hereinbelow) in a vicinity of (e.g., within 3 cm of, such as within 2 cm of) a greater palatine foramen 74 (labeled in FIG. 5, described hereinbelow). Second non-invasive SPG-stimulation electrode 20B is configured to be placed in oral cavity 72 in the vicinity of greater palatine foramen 74.

In the configurations shown in FIGS. 3A, 3B, 3C, 3E, and 3F, first and second SPG-stimulation sites 22A and 22B are ipsilateral, while in the configuration shown in FIG. 3D, first and second SPG-stimulation sites 22A and 22B are contralateral. The configurations of FIGS. 3C, 3E, and 3F may similarly be implemented contralaterally, rather than ipsilaterally. Contralateral stimulation may be applied bilaterally, i.e., with two current paths, each of which connects contralateral pairs of electrodes.

For some of these applications, system 10 further comprises an oral support 70, which is coupled to second non-invasive SPG-stimulation electrode 20B. Oral support 70 is configured to non-invasively apply second non-invasive SPG-stimulation electrode 20B to second SPG-stimulation site 22B when oral support 70 is placed within oral cavity 72.

For some of these applications, such as shown in FIGS. 4A-B and 3C, oral support 70 comprises a dental guard 76 configured to cover maxillary teeth 77 (labeled in FIG. 3C). Dental guard 76 is configured to non-invasively apply second non-invasive SPG-stimulation electrode 20B to second SPG-stimulation site 22B when dental guard 76 is placed on maxillary teeth 77.

Optionally, dental guard 76 is shaped is shaped so as to define one or two inward extensions 79, which support one or two second non-invasive SPG-stimulation electrodes 20B, respectively. Inward extensions 79 may help better position the one or two second non-invasive SPG-stimulation electrodes in the vicinity of left and right greater palatine foramens 74, respectively.

For others of these applications, such as shown in FIGS. 4C and 3B, oral support 70 comprises a gingival support band 78. Gingival support band 78 is configured to non-invasively apply second non-invasive SPG-stimulation electrode 20B to second SPG-stimulation site 22B when gingival support band 78 is placed between mucosa of bone 80 supporting maxillary teeth 77 and labial mucosa. It is noted that second SPG-stimulation site 22B is on opposite sides of bone 80 supporting maxillary teeth 77 in the configurations of FIGS. 4A-B and 3C, on the one hand, and FIGS. 4C and 3B, on the other hand (outer vs. inner sides of the bone).

For still others of these applications, oral support 70 comprises a maxillary orthodontic retainer 81. The maxillary orthodontic retainer is configured to non-invasively apply second non-invasive SPG-stimulation electrode 20B to second SPG-stimulation site 22B when the maxillary orthodontic retainer is coupled to maxillary teeth 77. Maxillary orthodontic retainer 81 may be removable, permanent, or a comprise both removable and permanent components (e.g., one or more small permanent components to which the main body of the retainer is removably couplable and decouplable, e.g., comprising magnetic couplers).

Reference is again made to FIGS. 3A-E. Oral support 70 (e.g., gingival support band 78, such as shown in FIGS. 3A-B and 3D-E, or dental guard 76, such as shown in FIG. 3C) holds second non-invasive SPG-stimulation electrode 20B within oral cavity 72 in the vicinity of greater palatine foramen 74 (labeled in FIG. 5, described hereinbelow), and first non-invasive SPG-stimulation electrode 20A is disposed on the external surface of skin 27 between mandibular notch 50 and zygomatic process 52 of temporal bone 54, such as by head support 38. Driving of alternating current by circuitry 32 between first and second non-invasive SPG-stimulation electrodes 20A and 20B at these respective locations generally causes the alternating current to pass through an SPG 84 (the left or right SPG, depending on the side of the head to which the alternating current is applied).

For some applications, first SPG-stimulation site 22A and greater palatine foramen 74 are ipsilateral.

Reference is still made to FIGS. 1A-B, 3A-E, and 4A-D. As mentioned above, for some applications, second SPG-stimulation electrode 20B is non-invasively applied to second SPG-stimulation site 22B. For some of these applications, second SPG-stimulation site 22B is within oral cavity 72 in a vicinity of greater palatine foramen 74 (labeled in FIG. 5, described hereinbelow), and non-invasively applying second SPG-stimulation electrode 20B to second SPG-stimulation site 22B comprises placing second SPG-stimulation electrode 20B in oral cavity 72 in the vicinity of greater palatine foramen 74.

Reference is now made to FIG. 4E, which is a schematic illustration of an oral support 200, in accordance with an application of the present invention. In this configuration, system 10 comprises at least first and second non-invasive SPG-stimulation electrodes 220A and 220B, instead of or in addition to first and second non-invasive SPG-stimulation electrodes 20A and 20B, described hereinabove. Oral support 200 is coupled to first and second non-invasive SPG-stimulation electrodes 220A and 220B, and configured to non-invasively apply first and second non-invasive SPG-stimulation electrodes 220A and 220B to respective first and second SPG-stimulation sites 222A and 222B when oral support 200 is placed within oral cavity 72. First and second SPG-stimulation sites 222A and 222B are on a hard palate 230 in the vicinity of a greater palatine nerve 232.

Circuitry 32 is configured to drive, between first and second non-invasive SPG-stimulation electrodes 220A and 220B, alternating current (AC) configured to increase cerebral blood flow (CBF) of the subject (and, typically, promotes vasodilation) by stimulating greater palatine nerve 232, and thus indirectly stimulating SPG 84. (Greater palatine nerve 232 is an extension of SPG 84.)

For some applications, oral support 200 comprises two sets of first and second non-invasive SPG-stimulation electrodes 220A and 220B, on opposite lateral sides of the oral support (configuration not shown). Circuitry 32 is configured to drive separate AC currents between the respective electrodes of each set, so as to stimulate left and right greater palatine nerves 232.

For example, a distance between first and second non-invasive SPG-stimulation electrodes 220A and 220B may be at least 6 mm, no more than 12 mm, and/or 6-12 mm.

For example, each of first and second non-invasive SPG-stimulation electrodes 220A and 220B may have a greatest dimension of at least 2 mm, no more than 6 mm (e.g., no more than 5 mm), and/or 2-6 mm, e.g., 2-5 mm.

For some applications, oral support 200 comprises a maxillary orthodontic retainer 202. The maxillary orthodontic retainer is configured to non-invasively apply first and second non-invasive SPG-stimulation electrodes 220A and 220B to respective first and second SPG-stimulation sites 222A and 222B when the maxillary orthodontic retainer is coupled to maxillary teeth 77. Maxillary orthodontic retainer 202 may be removable, permanent, or a comprise both removable and permanent components (e.g., one or more small permanent components to which the main body of the retainer is removably couplable and decouplable, e.g., comprising magnetic couplers).

For other applications (configuration not shown, but similar to the configuration shown in FIG. 4B), oral support 200 comprises a dental guard configured to cover maxillary teeth 77. The dental guard is configured to non-invasively apply first and second non-invasive SPG-stimulation electrodes 220A and 220B to respective first and second SPG-stimulation sites 222A and 222B when the dental guard is placed on maxillary teeth. Typically, the dental guard is shaped is shaped so as to define one or two inward extensions, which are similar to inward extensions 79 described hereinabove with reference to FIG. 4B. Each of the one or two inward extensions supports first and second non-invasive SPG-stimulation electrodes 220A and 220B. The inward extensions may help better position first and second non-invasive SPG-stimulation electrodes 220A and 220B in the vicinity of greater palatine nerve 232.

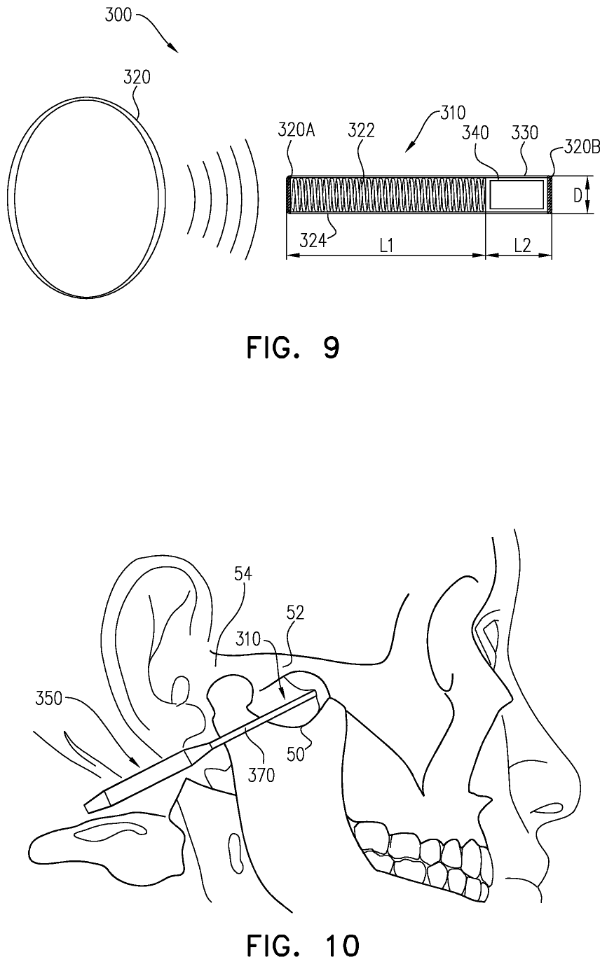

Reference is again made to FIGS. 1C-D, and is further made to FIG. 9, which is a schematic illustration of a neurostimulation system 300 comprising a neurostimulator 310, in accordance with an application of the present invention.

Reference is still further made to FIG. 10, which is a schematic illustration of a method of implanting neurostimulator 310, in accordance with an application of the present invention.

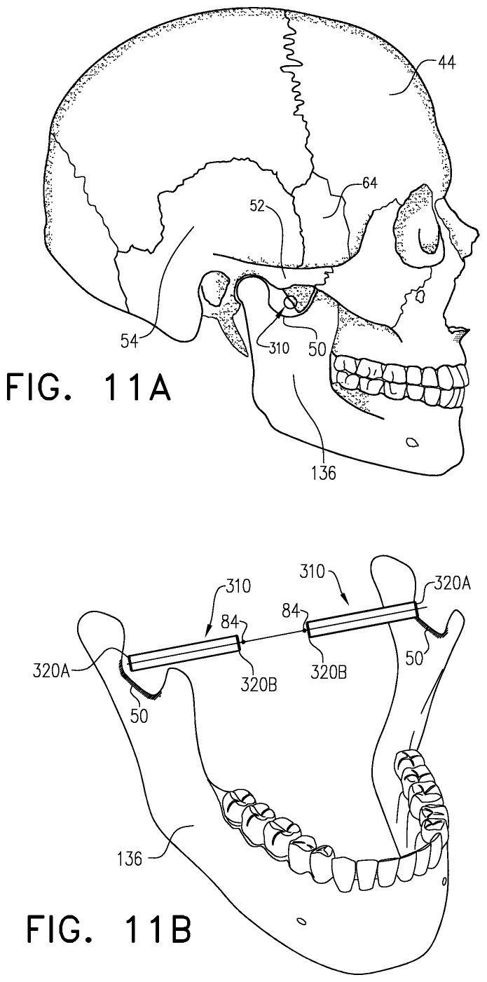

Reference is additionally made to FIGS. 11A-B, which are schematic illustrations showing neurostimulator 310 implanted in the face of the subject, in accordance with an application of the present invention. FIG. 11B shows two neurostimulators 310 implanted in left and right sides of the face, for stimulation of left and right SPGs 84, respectively.

Neurostimulation system 300 may be implemented as part of system 10, such as shown in FIGS. 1C-D, in which case system 10 may implement any of the features of the system described herein. Alternatively, neurostimulation system 300 is implemented independently of system 10.

As described above, for some applications, the first and the second SPG-stimulation electrode comprise implantable (and thus invasive) first and second SPG-stimulation electrodes 320A and 320B. Neurostimulator 310 comprises first and second SPG-stimulation electrodes 320A and 320B.

Neurostimulator 310 is configured to be inserted into a face of a subject (at an insertion site 312) and advanced between mandibular notch 50 and zygomatic process 52 of temporal bone 54 toward SPG 84. (For clarity of illustration, insertion site 312 is labeled in FIGS. 1C and 1D, even though the site is typically not visible after skin 27 has healed after insertion of the neurostimulator, and the site is covered by wireless transmitter 360 in FIG. 1C.)

In addition, such as shown in FIG. 10, in an application of the present invention, a method is provided comprising inserting neurostimulator 310 into the face of the subject and advancing neurostimulator 310 between mandibular notch 50 and zygomatic process 52 of temporal bone 54 toward SPG 84, the subject identified as at risk of or suffering from dementia, e.g., Alzheimer's disease. For example, neurostimulator 310 may be inserted by injecting neurostimulator 310 through skin 27 of the face, such as using an introducer tool 350, which may comprise a needle, such as shown in FIG. 10. Optionally, the insertion procedure is guide by ultrasound imaging.

For some applications, neurostimulator 310 is advanced toward SPG 84 until a distance between neurostimulator 310 and SPG 84 is 0-3 mm.

For some applications, once neurostimulator 310 is tentatively positioned at the target site, an intra-procedural step is performed in which neurostimulator 310 is activated in order to verify that the applied stimulation is effective. If not, the neurostimulator may be repositioned and the intra-procedural step repeated. Once the effectiveness of the stimulation is confirmed, neurostimulator 310 is detached from introducer tool 350.

Typically, circuitry 32 is configured to activate neurostimulator 310 to drive, between first and second SPG-stimulation electrodes 320A and 320B, alternating current configured to increase CBF of the subject by stimulating SPG 84. The alternating current may optionally have any of the parameters of the SPG-stimulation alternating current described hereinbelow.

For some applications, such as shown in FIG. 9, neurostimulator 310 comprises a housing 330, which may, for example, be cylindrical, e.g., having a length of 5-15 mm, e.g., 10 mm (L1+L2) and/or an external diameter D of 1-2 mm, e.g., 1.5 mm. Optionally, first and second SPG-stimulation electrodes 320A and 320B are disposed at opposite ends of housing 330, such as shown. Optionally, housing 330 is coated with a biocompatible coating, such as parylene and titanium nitrate, optionally applied as several layers of parylene and titanium nitrate.

For some applications, neurostimulator 310 comprises an antenna coil 322. Optionally, antenna coil 322 is helical. For example, antenna coil 322 may have a length L1 of 3-15 mm, e.g., 7 mm. Optionally, neurostimulator 310 further comprises a magnetic core around which antenna coil 322 is wound; for example, the magnetic core may comprise ferrite.

For some applications, neurostimulator 310 comprises implant circuitry 340, which is configured to wirelessly receive energy using antenna coil 322 and apply a signal to SPG 84 using the energy. Optionally, neurostimulator 310 comprises a power storage unit, e.g., comprising one or more capacitors and/or a rechargeable battery. Optionally, implant circuitry 340 has a length L2 of 2-4 mm, e.g., 3 mm.

Alternatively, neurostimulator 310 does not comprise any active electronic components, and instead passively applies the received energy to SPG 84.

For some applications, circuitry 32 is configured to configure first and second SPG-stimulation electrodes 320A and 320B as an anode and a cathode, respectively. For example, this configuration may be useful in configurations in which only the positive portion or the negative portion of the AC wave is applied.

For some applications, neurostimulation system 300 further comprises a wireless transmitter 360, schematically labeled in FIGS. 1C and 1D. Typically, wireless transmitter 360 comprises a coil 320, shown schematically in FIG. 9. Circuitry 32 is configured to drive wireless transmitter 360 to wirelessly transmit energy to antenna coil 322 of neurostimulator 310 from outside the face. For example, circuitry 32 may drive the transmitter to transmit the energy at a frequency of 6-14 MHz, e.g., 6.78 MHz or 13.56 MHz.

Reference is made to FIGS. 9 and 10. For some applications, antenna coil 322 is disposed closer to a first end of neurostimulator 310 than to a second end opposite the first end, and neurostimulator 310 is inserted to the face such that the first end of neurostimulator 310 is more superficial (closer to skin 27) than the second end, in order to minimize the distance between wireless transmitter 360 and antenna coil 322.

For some applications, neurostimulation system 300 (and/or system 10) comprises head support 38, described hereinabove. For some applications, such as shown in FIG. 1C, head support 38 is configured to apply wireless transmitter 360 to the external surface of skin 27 on the face of the subject. For some applications, head support 38 comprises headphone 40, described hereinabove. For some of these applications, headphone 40 comprises a transmitter support arm 325, to which wireless transmitter 360 is coupled. For other applications, wireless transmitter 360 is disposed within one of the one or two earphones 42, such as shown in FIG. 1D. (In configurations in which both SPGs 84 are stimulated, typically two wireless transmitters 360 are provided, which are either coupled to headphone 40 by two respective support arms 325, or disposed within the two earphones 42, respectively.)

Reference is made to FIGS. 1A-B, 1C-D, 2A-B, 3A-F, and 4A-E. For some applications, circuitry 32 is configured to drive the alternating current in pulses having a pulse width typically of at least 50 microseconds, such as at least 100 microseconds (e.g., at least 200 microseconds), no more than 5,000 microseconds, such as no more than 600 microseconds (e.g., no more than 400 microseconds), and/or 50-5,000 microseconds, e.g., 100-600 microseconds, such as 200-400 microseconds and/or a frequency of typically at least 10 Hz, no more than 100 Hz (e.g., no more than 40 Hz), and/or 10-100 Hz, such as 10-60 Hz, e.g., 10-40 Hz.

The SPG stimulation may increase levels of nitric oxide (NO) in the brain, which may promote vasodilation and/or have a positive effect on the metabolism of amyloid beta.

For any of the applications described herein, circuitry 32 may be configured to drive the alternating current as a strength that is insufficient to induce a significant increase in permeability of a blood-brain barrier (BBB) of the subject.

Optionally, circuitry 32 is configured to ramp up the applied alternating current during an initial period after activation, e.g., with a duration of 1 minute.

Reference is now made to FIG. 5, which is a schematic illustration of the insertion of a wire 90 into a greater palatine canal 92, in accordance with an application of the present invention. In this application, system 10 further comprises wire 90. Wire 90, including both ends 94A and 94B thereof, is configured to be minimally invasively inserted into greater palatine canal 92 via greater palatine foramen 74. Typically, wire 90 is insulated except at respective end portions including ends 94A and 94B. Typically, wire 90 has a length of at least 1 cm, no more than 3 cm, and/or 1-3 cm, and/or a diameter of at least 0.5 mm, no more than 2 mm, and/or 0.5-2 mm.

For some applications, any of the methods described hereinabove further comprise minimally invasively inserting wire 90, including both ends 94A and 94B thereof, into greater palatine canal 92 via greater palatine foramen 74. Optionally, a mucoperiosteal incision is made before or during the insertion. Second SPG-stimulation electrode 20B is placed in oral cavity 72 in the vicinity of greater palatine foramen 74 such that second SPG-stimulation electrode 20B is separated from the wire by masticatory mucosa. As a result, current passes between the area of mandibular notch 50 on one side of head 44 and greater palatine foramen 74 in oral cavity 72 on the same side of head 44 through the wire 90 (which provides the path of least resistance between first and second non-invasive SPG-stimulation electrodes 20A and 20B).

tDCS

Reference is again made to FIGS. 1A-B, and is additionally made to FIGS. 6A-B, which are schematic illustrations of application of first and second non-invasive tDCS electrodes 24A and 24B, in accordance with respective applications of the present invention.

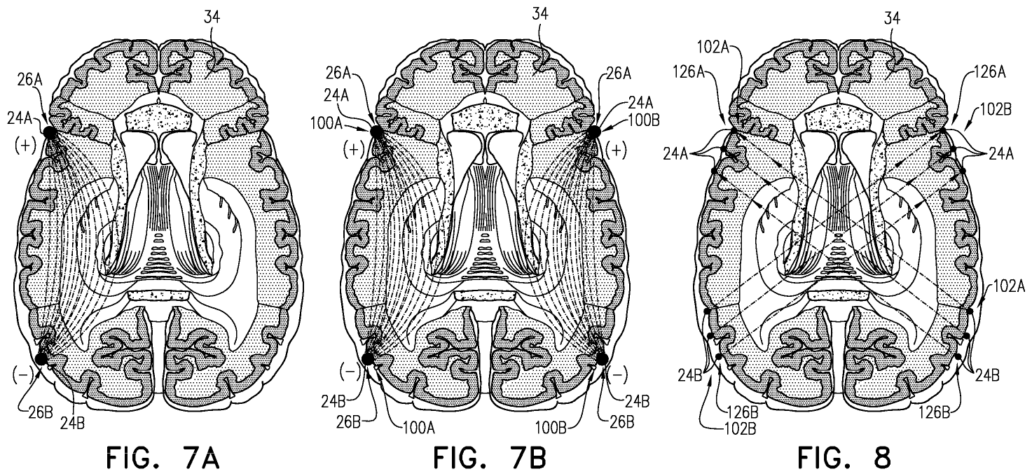

Reference is further made to FIGS. 7A-B, which are schematic illustrations of direct current paths between first and second non-invasive tDCS electrodes 24A and 24B, in accordance with respective applications of the present invention. (For clarity of illustration, electrode leads and circuitry 32 are not shown in FIG. 6A-B or 7A-C.)

Reference is still further made to FIG. 8, which is a schematic illustration of direct current paths between two sets of first and second non-invasive tDCS electrodes 24A and 24B, respectively, in accordance with an application of the present invention.

As described above, in some applications, circuitry 32 is configured to drive, between first and second non-invasive tDCS electrodes 24A and 24B, direct current configured to promote amyloid beta clearance from brain 34. This tDCS generally mostly impacts the temporal lobe, where the hippocampus and entorhinal cortex are located.

In some applications of the present invention, head support 38 is configured to apply:
first non-invasive tDCS electrode 24A to the external surface of skin 27 at first tDCS site 26A on a temple 56 of the subject, and/or
second non-invasive tDCS electrode 24B to the external surface of skin 27 at second tDCS site 26B at a non-cephalic site (i.e., a site not on head 44 of the subject).

For example, the non-cephalic site may be located on of a part of the subject selected from the group consisting of: neck 58 (e.g., a nape 62 of neck 58 or a side of neck 58 under the ear) and shoulder 60 (e.g., a portion of shoulder 60 near neck 58); for example, second tDCS site 26B may be over a trapezius muscle or over a deltoid muscle.

Driving of the direct current by circuitry 32 between first and second tDCS electrodes 24A and 24B may enhance microglial cell activation to promote degradation and clearance of amyloid beta, such as by increasing the membrane potential in glial cells, i.e., depolarizing the membrane, which results in activation/modulation of glial activity. In addition, driving of the direct current by circuitry 32 between first and second tDCS electrodes 24A and 24B may promote amyloid beta clearance from brain 34 (e.g., parenchyma of brain 34) towards the positive electrode. For example, the direct current may help clear amyloid beta from parenchyma because of the opposite polarity of current and the amyloid beta. Optionally, driving the direct current alternatively or additionally promotes clearance of other metabolic waste from brain 34. In addition, driving of the direct current by circuitry 32 between first and second tDCS electrodes 24A and 24B may depolarize the membrane potential of neurons, thereby increasing the sensitivity of the neurons and increasing the likelihood of action potentials, which may increase the sensitivity the neurons to gamma band entrainment stimulation, as described herein.

As used in the present application, including in the claims and Inventive Concepts, a “temple” is the region of the head behind the eyes and in front of the ear, overlying a portion of temporal bone 54 and a portion of a sphenoid bone 64. For some applications, first non-invasive tDCS electrode 24A is placed at first tDCS site 26A overlying a portion of sphenoid bone 64, at least in part because the sphenoid bones are the thinnest area of the skull and as such has low resistance and high current transfer towards the brain parenchyma. For other applications, first non-invasive tDCS electrode 24A is placed at first tDCS site 26A overlying a portion of temporal bone 54, or overlying portions of both the temporal bone and the sphenoid bone. This placement of the electrode over sphenoid bone 64 or temporal bone 54 may target the temporal lobe where the hippocampus and entorhinal cortex reside. These are the areas most sensitive to amyloid beta aggregations. For some applications, first tDCS site 26A is closer to the ear than to the eye.

For some applications, first tDCS site 26A is, by way of example and not limitation, at FT10 (for the right side) or FT9 (for the left side), in accordance with the 10-10 electrode system for EEG recording, as is well known in the EEG art. Alternatively, for some applications, first tDCS site 26A is, by way of example and not limitation, at F8, F10, FT8, FT10, or T8 (for the right side) or F7, F9, FT7, FT9, or T7 (for the left side).

For some applications, circuitry 32 is configured to configure first non-invasive tDCS electrode 24A to be an anode and second non-invasive tDCS electrode 24B to be a cathode.

For some applications, circuitry 32 is configured to drive the direct current at an amplitude of 1-5 milliamps, e.g., 2-4 milliamps.

Optionally, circuitry 32 is configured to ramp up the applied direct current during an initial period after activation, e.g., with a duration of up to one minute.

For some applications, circuitry 32 is configured to drive the direct current in treatment sessions having a duration of 15-60 minutes, such as 20-40 minutes, e.g., 30 minutes.

For some applications, system 10 is configured to apply the tDCS at different times to the two sides of the head, while for other applications system 10 is configured to apply the tDCS simultaneously to both sides of the head.

Reference is made to FIGS. 1A-B. In some applications of the present invention, a method is provided that comprises:
non-invasively applying first non-invasive tDCS electrode 24A to first tDCS site 26A on the external surface of skin 27 of temple 56 of a subject identified as at risk of or suffering from dementia, e.g., Alzheimer's disease;
non-invasively applying second non-invasive tDCS electrode 24B to second tDCS site 26B on the external surface of skin 27 at a non-cephalic site; and
activating circuitry to drive, between first and second non-invasive tDCS electrodes 24A and 24B, direct current configured to promote amyloid beta clearance from brain 34 of the subject.

For example, the non-cephalic site may be located on a part of the subject selected from the group consisting of: neck 58 (e.g., nape 62 of neck 58 or a side of neck 58 under the ear) and shoulder 60 (e.g., a portion of shoulder 60 near neck 58); for example, second tDCS site 26B may be over a trapezius muscle or over a deltoid muscle.

The above-mentioned locations of second tDCS site 26B are relatively far from brain 34, which may help avoid stimulation of neurons in the brain (particularly in configurations in which second non-invasive tDCS electrode 24B is configured as a cathode).

For some applications, such as shown in FIG. 7A, first and second tDCS sites 26A and 26B are ipsilateral, in which case the tDCS is applied to the lobe of the brain on the side of the head at which the electrodes are applied. The tDCS typically has a clinically-relevant impact on the hippocampus and the entorhinal cortex.

Optionally, second non-invasive tDCS electrode 24B (on neck 58 or shoulder 60) is larger than first non-invasive tDCS electrode 24A (on temple 56).

For some applications, such as shown in FIG. 7B, tDCS is applied bilaterally, i.e., to both lobes of the brain, either in series or simultaneously, using a first set 100A of electrodes applied to left first and second tDCS sites 26A and 26B, and a second set 100B of electrodes applied to right first and second tDCS sites 26A and 26B, such as at the respective locations described hereinabove.

For some applications, such as shown in FIG. 8, first and second tDCS sites 26A and 26B are contralateral. Optionally, a first set 102A of electrodes is applied to a left first tDCS site 126A and a right second tDCS site 126B, and a second set 102B of electrodes is applied to a right first tDCS site 126A and a left second tDCS site 126B, such as at the respective locations described hereinabove.

For some applications, first and second non-invasive tDCS electrodes 24A and 24B are positioned such that the tDCS is applied to the hippocampus.

For some applications, first and second non-invasive tDCS electrodes 24A and 24B are relatively small, e.g., have a diameter of 0.5-2 mm, which may facilitate focusing the direct current direction with increased accuracy.

For some applications, the subject is encouraged (by system 10 and/or by a healthcare worker or assistant) to perform cognitive activity during application of the tDCS. Such cognitive activity may be beneficial for treatment.

### Gamma Band Entrainment Stimulation

As described above, in some applications, system 10 comprises one or more gamma band entrainment stimulators 28, and circuitry 32 is configured to drive the one or more gamma band entrainment stimulators 28 to apply to the subject gamma band entrainment stimulation that modulates oscillatory brain activity of the subject.

Also as described above, for some applications, the one or more gamma band entrainment stimulators 28 comprise one or more speakers 29, configured to applied to one or two ears 30 of the subject. Circuitry 32 is configured to drive the one or more speakers 29 to apply, to the one or two ears 30, auditory gamma band entrainment stimulation that modulates the oscillatory brain activity. This auditory gamma band entrainment stimulation may cause a slowdown in brain shrinkage and ventricle enlargement, an increase in EEG gamma power, an in increase in blood perfusion, and/or cognitive improvement. It may also modulate microglia activity, reduce tau burden, and reduce amyloid beta load. Evidence of the clinical benefit of auditory gamma band entrainment stimulation is provided, for example, in the following articles: McDermott B et al. 2018 (cited hereinabove in the Background section), and Martorell A J et al., “Multi-sensory Gamma Stimulation Ameliorates Alzheimer's-Associated Pathology and Improves Cognition,” Cell. 2019 Apr. 4; 177(2):256-271.

Optionally, the one or more gamma band entrainment stimulators 28 are configured to vibrate in order to apply and/or enhance the gamma band entrainment.

For some applications, circuitry 32 is configured to drive the one or more speakers 29 of the one or two earphones 42 to apply the auditory stimulation at a frequency of 30-60 Hz, e.g., 40 Hz. For example, circuitry 32 may be configured to vary the frequency around a midpoint in order to avoid the mechanoreceptors becoming habituated and unresponsive (e.g., vary from 39.96 Hz to 40.06 Hz).

For some applications, the one or more gamma band entrainment stimulators 28 comprises one or more somatosensory stimulators, and circuitry 32 is configured to drive the one or more somatosensory stimulators to apply somatosensory gamma band entrainment stimulation that modulates the oscillatory brain activity. For example, the one or more somatosensory stimulators may comprise vibratory stimulators. Evidence of the effect of vibratory stimulation on gamma band entrainment is provided, for example, by Suk H J et al., “Vibrotactile stimulation at gamma frequency mitigates pathology related to neurodegeneration and improves motor function,” Front Aging Neurosci. 2023 May 18; 15:1129510.

For some applications, circuitry 32 is configured to cause vibration of at least one of first and second non-invasive tDCS electrodes 24A and 24B at a frequency of 30-60 Hz (typically 40 Hz). This vibration may modulate oscillatory brain activity, as discussed above. Alternatively or additionally, this vibration may reduce impedance of the stratum corneum of skin 27, thereby increasing the efficacy of the tDCS. For some applications, system 10 is configured to measure impedance of skin 27, and commence tDCS when the impedance falls below a threshold level. For example, the threshold level may be 1-10 kiloohm.

For some applications, the one or more gamma band entrainment stimulators 28 comprise two or more non-invasive transcranial Direct Alternating Stimulation (tACS) electrodes, and circuitry 32 is configured to drive the two or more non-invasive tACS electrodes to apply tACS gamma band entrainment stimulation that modulates the oscillatory brain activity.

The tACS may increases EEG gamma power, increase blood perfusion, modulate microglia activity, improve cognitive abilities, reduce tau burden, and/or reduce amyloid beta load.

Optionally, the one or more gamma band entrainment stimulators 28 are configured to vibrate in order to apply and/or enhance the tACS gamma band entrainment.

Evidence of the clinical efficacy of tACS is provided, for example, in the following articles:
Benussi A et al., “Exposure to gamma tACS in Alzheimer's disease: A randomized, double-blind, sham-controlled, crossover, pilot study,” Brain Stimul. 2021 May-June; 14(3):531-540
Dhaynaut M et al., “Impact of 40 Hz Transcranial Alternating Current Stimulation on Cerebral Tau Burden in Patients with Alzheimer's Disease: A Case Series,” J Alzheimers Dis. 2022; 85(4): 1667-1676
Manippa V et al., “Cognitive and Neuropathophysiological Outcomes of Gamma-tACS in Dementia: A Systematic Review,” Neuropsychol Rev. 2023 Mar. 6

For some applications, circuitry 32 is configured to drive the two or more non-invasive tACS electrodes to apply tACS at a frequency of 30-60 Hz (typically 40 Hz).

The tACS may be applied to both temporal lobes separately or simultaneously (bi-lateral stimulation), or to a single temporal lobe.

For some applications, the one or more gamma band entrainment stimulators 28 comprise one or more non-invasive light sources, and circuitry 32 is configured to drive the one or more non-invasive light sources to apply non-invasive light flicker gamma band entrainment stimulation that modulates the oscillatory brain activity. Evidence of the efficacy of such stimulation in mice is provided, for example, in Iaccarino H F et al., “Gamma frequency entrainment attenuates amyloid load and modifies microglia,” Nature. 2016 Dec. 7; 540(7632):230-235.

For some applications, gamma band entrainment is applied for a total duration of 30-90 minutes daily, such as 60 minutes daily.

For some applications, system 10 is configured to apply two or more types of gamma band entrainment stimulation.

### Additional Optional Features

For some applications, system 10 comprises an integrated EEG capability for assessing brain stimulation in real time (such as for safety and efficacy).

For some applications, software is used to define the optimal electrode configuration and placement. The software leverages the individual subject's MRI and/or CT head images (such as including hippocampus location coordinates) and provides detailed guidelines regarding where to place the device on the head and/or which subset of electrodes to activate during treatment to achieve optimal therapeutic results.

For some applications, the treatment regimen is adjusted according to levels of amyloid beta concentration as seen in PET imaging (the more plaques, the more aggressive the regimen).

For some applications, system 10 comprises a patient application, which allows the subject to control certain aspects of the system device, and which provides treatment statistics and treatment reminders. Typically, the patient application is connected to the cloud in order to allow caregivers and physicians to monitor device usage and compliance. Alternatively or additional, remote-control software is provided to allow remote activation/deactivation and/or control of the system.

For some applications, treatment or prevent ion using system 10 is combined with drug therapies, which may enable a reduced drug dose by accelerating clearance effect of drugs targeting amyloid beta or neurofibrillary tau tangles. Exemplary drugs include Adulham and Leqimbi, Crenezumab, E2609, Gantenerumab, and Solanezumab.

Although the stimulation techniques described herein have been described as treating and/or preventing Alzheimer's disease, these techniques may also be used for treatment and/or prevention of other types of dementia, such as vascular dementia and Lewy body dementia.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method comprising:
inserting a neurostimulator through an external surface of a side of a face of a subject and between a mandibular notch on the side of the face and a zygomatic process of a temporal bone on the side of the face, and thereafter advancing the neurostimulator toward a sphenopalatine ganglion (SPG) on the side of the face; and
increasing cerebral blood flow (CBF) of the subject by activating the neurostimulator to drive, between first and second SPG-stimulation electrodes of the neurostimulator, alternating current configured to stimulate the SPG.

2. The method according to claim 19,
wherein the dementia is Alzheimer's disease, and
wherein inserting the neurostimulator comprises inserting the neurostimulator through the external surface of the side of the face of the subject identified as at risk of or suffering from Alzheimer's disease.

3. The method according to claim 1, wherein inserting the neurostimulator comprises injecting the neurostimulator through skin of the external surface of the side of the face.

4. The method according to claim 1, wherein activating the neurostimulator comprises activating the neurostimulator to drive the alternating current in pulses having a pulse width of 50-5,000 microseconds and a frequency of 10-60 Hz.

5. The method according to claim 1, wherein activating the neurostimulator comprises activating the neurostimulator to drive the alternating current at a frequency of 10-60 Hz.

6. The method according to claim 1, wherein activating the neurostimulator comprises activating the neurostimulator to drive the alternating current in pulses having a pulse width of 50-5,000 microseconds.

7. The method according to claim 1,
wherein the neurostimulator comprises an antenna coil, and
wherein activating the neurostimulator comprises wirelessly transmitting energy to the antenna coil from outside the face.

8. The method according to claim 7,
wherein the antenna coil is disposed closer to a first end of the neurostimulator than to a second end opposite the first end, and
wherein inserting the neurostimulator comprises inserting the neurostimulator through the external surface of the side of the face such that the first end of the neurostimulator is more superficial than the second end.

9. The method according to claim 7, wherein wirelessly transmitting the energy comprises:
coupling a head support to a head of the subject, the head support including circuitry; and
activating the circuitry to drive a wireless transmitter to wirelessly transmit the energy to the antenna coil of the neurostimulator.

10. The method according to claim 9, wherein the head support is configured to apply the wireless transmitter to an external surface of skin on the face of the subject.

11. The method according to claim 9, wherein the head support comprises a headphone, which comprises one or two earphones, and wherein the wireless transmitter is disposed within one of the one or two earphones.

12. The method according to claim 9,
wherein the head support comprises a headphone, which comprises one or two earphones, which comprise one or more speakers,
wherein coupling the head support to the head comprises coupling the headphone to the head such that the headphone positions the one or two earphones over or in one or two respective ears of the subject, and
wherein activating the circuitry comprises activating the circuitry to drive the one or more speakers of the one or two earphones to apply, to the one or two ears, auditory stimulation that modulates oscillatory brain activity of the subject.

13. The method according to claim 12, wherein activating the circuitry comprises activating the circuitry to drive the one or more speakers of the one or two earphones to apply the auditory stimulation at a frequency of 30-60 Hz.

14. The method according to claim 9,
wherein the head support is coupled to first and second non-invasive transcranial Direct Current Stimulation (tDCS) electrodes,
wherein coupling the head support to the head comprises coupling the head support to the head such that the head support applies:
the first non-invasive tDCS electrode to an external surface of skin at a first tDCS site on a temple of the subject, and
the second non-invasive tDCS electrode to the external surface of the skin at a non-cephalic second tDCS site, and
wherein activating the circuitry comprises activating the circuitry to drive, between the first and the second non-invasive tDCS electrodes, direct current configured to promote amyloid beta clearance from a brain of the subject.

15. The method according to claim 14, wherein coupling the head support to the head comprises coupling the head support to the head such that the head support applies the non-cephalic second non-invasive tDCS electrode to the external surface of the skin on a part of the subject selected from the group consisting of: a neck and a shoulder.

16. The method according to claim 15, wherein coupling the head support to the head comprises coupling the head support to the head such that the head support applies the non-cephalic second non-invasive tDCS electrode to the external surface of a nape of the subject.

17. The method according to claim 14, wherein activating the circuitry comprises activating the circuitry to drive the direct current at an amplitude of 1-5 milliamps.

18. The method according to claim 1, further comprising:
non-invasively applying first and second non-invasive transcranial Direct Current Stimulation (tDCS) electrodes to an external surface of skin at first and second tDCS sites, respectively; and
activating circuitry to:
activating the neurostimulator to drive the alternating current between the first and second SPG-stimulation electrodes,
drive, between the first and the second non-invasive tDCS electrodes, direct current configured to promote amyloid beta clearance from a brain of the subject, and
apply to the subject gamma band entrainment stimulation that modulates oscillatory brain activity of the subject.

19. The method according to claim 1, wherein inserting the neurostimulator comprises inserting the neurostimulator through the external surface of the side of the face of the subject identified as at risk of or suffering from dementia.
